SEQUENCE LISTING (20 sequences)

SEQ ID NO: 1 - protein, 222 aa, Homo sapiens
MEPHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 2 - protein, 4 aa, Artificial Sequence
  synthetic
RSKR

SEQ ID NO: 3 - protein, 4 aa, Artificial Sequence
  synthetic
  misc_feature (3)..(3): Xaa can be any naturally occurring amino acid
VSXR

SEQ ID NO: 4 - protein, 4 aa, Artificial Sequence
  synthetic
  misc_feature (1)..(1): Xaa can be any naturally occurring amino acid
  misc_feature (3)..(3): Xaa can be any naturally occurring amino acid
XSXR

SEQ ID NO: 5 - protein, 4 aa, Artificial Sequence
  synthetic
  misc_feature (1)..(1): Xaa can be any naturally occurring amino acid
XSAR

SEQ ID NO: 6 - protein, 4 aa, Artificial Sequence
  synthetic
VSAR

SEQ ID NO: 7 - protein, 4 aa, Artificial Sequence
  synthetic
  misc_feature (2)..(2): Xaa can be any naturally occurring amino acid
RXKR

SEQ ID NO: 8 - protein, 4 aa, Artificial Sequence
  synthetic
  misc_feature (1)..(3): Xaa can be any naturally occurring amino acid
XXXR

SEQ ID NO: 9 - protein, 4 aa, Artificial Sequence
  synthetic
  misc_feature (2)..(2): Xaa can be any naturally occurring amino acid
VXAR

SEQ ID NO: 10 - DNA, 41 bp, Artificial Sequence
  synthetic
cttcaacaggactcacgtgagcgcgcgctcatcatcccatc

SEQ ID NO: 11 - DNA, 41 bp, Artificial Sequence
  synthetic
gatgggatgatgagcgcgcgctcacgtgagtcctgttgaag

SEQ ID NO: 12 - DNA, 101 bp, Artificial Sequence
  synthetic
agatctcgatcccgcgaaattaatacgactcactataggggaattgtgagcggataacaattcccctctagaaataattttgtttaactttaagaaggaga

SEQ ID NO: 13 - DNA, 116 bp, Artificial Sequence
  synthetic
tatacatatggctagcatgactggtggacagcaaatgggtcgcggatccggctgctaacaaagcccgaaaggaagctgagttggctgctgccaccgctgagcaataactagcataa

SEQ ID NO: 14 - protein, 16 aa, Artificial Sequence
  synthetic
MASMTGGQQMGRGSGC

SEQ ID NO: 15 - DNA, 78 bp, Artificial Sequence
  synthetic
ggtcgggatccggctgctaacaaagcccgaaaggaagctgagttggctgctgccaccgctgagtaataactagcataa

SEQ ID NO: 16 - protein, 22 aa, Artificial Sequence
  synthetic
GRDPAANKARKEAELAAATAEQ

SEQ ID NO: 17 - DNA, 77 bp, Artificial Sequence
  synthetic
ggtcggatccggctgctaacaaagcccgaaaggaagctgagttggctgctgccaccgctgagcaataactagcataa

SEQ ID NO: 18 - protein, 23 aa, Artificial Sequence
  synthetic
GRIRLLTKPERKLSWLLPPLSNN

SEQ ID NO: 19 - DNA, 13 bp, Artificial Sequence
  synthetic
taccatggctagc

SEQ ID NO: 20 - DNA, 39 bp, Artificial Sequence
  synthetic
ccccttggggcctctaaacgggtcttgaggggttttttg

(19) United States
(12) Patent Application Publication
(10) Pub. No.: US 2018/0086805 A1
(43) Pub. Date: Mar. 29, 2018

(54) proNGF mutants and uses thereof for the preparation of NGF mutants

(71) Applicant: Chiesi Farmaceutici S.p.A., Parma (IT)

(72) Inventors: Antonino CATTANEO, Roma (IT); Francesca MALERBA, Roma (IT)

(21) Appl. No.: 15/593,527

(22) Filed: May 12, 2017

Related U.S. Application Data
(60) Provisional application No. 62/339,337, filed on May 20, 2016.

(51) Int. Cl. C07K 14/48; C12P 21/06

(52) U.S. Cl. CPC C07K 14/48; C12Y 304/21004; C12P 21/06

(57) ABSTRACT

Pro-NGF—Nerve Growth Factor—mutants and their uses for the production of NGF mutants are disclosed. Also disclosed are useful new process to optimize and improve the purification of NGF P61SR100E, a therapeutic protein. The invention further provides a mutant pro-NGF wherein the protease cleavage site RASKRB is substituted at least at position RA and K by a non-basic amino acid, and the mutant pro-NGF further includes at least one mutation selected from P165S or R204E.

### CROSS-REFERENCE TO RELATED APPLICATIONS

This application claims the benefit of U.S. provisional patent application Ser. No. 62/339,337, filed May 20, 2016, the contents of which are herein incorporated by reference.

### TECHNICAL FIELD

The present invention relates to pro-NGF mutants and their uses, in particular for the production of NGF mutants.

### BACKGROUND ART

Nerve Growth Factor (NGF) was the first discovered member of the neurotrophin family, that regulates many functions of neuronal cells. NGF was originally identified for its developmental actions, as a neurotrophic survival factor necessary for the development and differentiation of sympathetic and sensory neurons during embryogenesis. In the adult, NGF was subsequently shown to exert pleiotropic actions in various neural and non-neural cells, including phenotypic maintenance of basal forebrain cholinergic neurons and functional modulation of sensory neurons. In addition to its neuronal targets, NGF has been shown to act on astrocytes and microglia, cells of the immune system such as mast cells and basophils, keratinocytes, blood vessel endothelial cells and many others. NGF exerts its effects in responsive cells by interacting with either one or both of two cell surface receptors: the tyrosine kinase TrkA and p75NTR.

For all these abilities NGF holds a great therapeutic promise for Alzheimer's disease, diabetic neuropathies, ophthalmic diseases and dermatological ulcers. However the therapeutic clinical applications of NGF have been hampered by its potent pro-nociceptive action.

A recombinant, painless human NGF R100E (hNGF R100E) mutant has been engineered, inspired by the R100W genetic mutation in the NGF gene, found in patients affected by a rare genetic congenital form of insensitivity to pain (HSAN V). The hNGF R100E mutant is equal to hNGF wt in neurotrophic potency but shows a lower nociceptive activity with respect to hNGF wt. Another mutation, P61S, has been inserted on the hNGF R100E mutant. The P61S mutation makes the NGF selectively detectable against wild type hNGF by a specific monoclonal antibody. Therefore, the painless double mutant hNGF P61S R100E shows the same neurotrophic activity of NGF wild type, a low nociceptive action and the traceability in biological samples. All these features make the painless mutant hNGF P61SR100E interesting for therapeutic usage (WO 2008/006983).

Recombinant NGF is expressed as proNGF in E. Coli. Mature NGF is obtained by a proteolytic cleavage in controlled conditions. The enzyme trypsin cleaves the protein substrates at exposed basic sites. In the case of proNGF, trypsin completely cuts the propeptide, leaving intact the mature part. This feature is due to the fact that proNGF is an intrinsically unstructured protein (Paoletti et al., 2009) and exposes its basics residues, while NGF has a well-defined tertiary structure.

The procedure has severe problems. Indeed, mass spectrometry and HPLC analyses (FIG. 1) demonstrate that trypsin gives raise to different cleavage products with similar molecular weights. Due to the proximity of 3 basic amino acids, the trypsin enzyme does not cut precisely at the sequence Arg-Ser-Lys-Arg (R-S-K-R, SEQ ID NO: 2), that is immediately upstream the first amino acid of mature NGF, and represents the consensus sequence for the endopeptidase furin. A non-homogenous product is not acceptable for proceeding in clinical trials, in which a GMP product is mandatory. Moreover, the proteolytic cleavage has shown a low efficiency: high amounts of the enzyme are necessary to obtain a complete cleavage.

Then there is the need for optimizing current expression and purification process in order to obtain: higher yields, a full reproducibility, and a molecularly homogenous product, adequate for the GMP process.

EP 2 672 984 discloses a method whereby, by performing a site directed mutagenesis, the furin consensus site R-S-K-R (SEQ ID NO: 2) at the C-terminal of wild-type proNGF is changed into V-S-A-R (SEQ ID NO: 6). This leaves only one basic residue and provides precision to trypsin cleavage.

### SUMMARY OF THE INVENTION

In the present invention the furin consensus site R-S-K-R (SEQ ID NO: 2) at the C-terminal of proNGF wild-type (WT) was changed into V-S-A-R (SEQ ID NO: 6, hereafter the VSAR method). The change was also applied to proNGF P61SR100E and to the single mutants proNGF P61S and proNGF R100E.

The mutants were expressed in E. coli and purified. A number of different cleavage protocols were tested, in order to obtain at the same time, a complete cleavage, a high yield of mature NGF, and the minimum concentration of trypsin used.

Surprisingly, the inventors found that the VSAR method is extremely sensitive to experimental conditions, and to mutations located in the mature NGF moiety. Testing a large number of different experimental conditions and running comparative analyses, the inventors found unexpectedly that the VSAR method is much more efficient with the double mutant proNGF P61SR100E than it is with wild type proNGF. This result could not have been predicted on the basis of what is known on the stability of WT NGF and of NGF P61SR100E. Indeed, the inventors started their experiments with the expectation and the prediction that the performance of the VSAR mutation, being located in the pro-domain that is cleaved off (and being the pro-domain an intrinsically unfolded domain), would not be affected by mutations located in the mature NGF part of the molecule. Moreover, since it is known that proNGF P61SR100E and NGF P61SR100E have a considerably lower stability (and higher sensitivity to trypsin degradation) than the WT counterparts (Malerba et al 2015), the inventors would have predicted that the VSAR method, in the context of the proNGF P61SR100E mutations, even if working, would provide lower yields.

The results obtained in the present invention are surprising and unexpected and provide a useful new process to optimize and improve the purification of NGF P61SR100E therapeutic protein.

Then, the present invention provides a mutant pro-NGF wherein the protease cleavage site R_{A}SKR_{B} (SEQ ID NO: 2) is substituted at least at position RA and K by a non-basic amino acid said mutant pro-NGF further comprising at least one mutation selected from P165S or R204E (corresponding to P61S and R100E in mature NGF).

Preferably the non-basic amino acid is selected from Alanine, Glycine, Valine, Serine, Threonine, Methionine, Tyrosine, Histidine, Asparagine, Aspartic Acid, Glutamine, Glutamic Acid, Phenylalanine, Isoleucine, Leucine, Tryptophan, Cysteine, and Proline.

Preferably the protease cleavage site has the sequence VSAR (SEQ ID NO: 6).

Preferably the mutant pro-NGF comprises both mutations P165S and R204E.

The invention also provides the mutant pro-NGF as defined above for use in a method to produce a mutant NGF.

Preferably the mutant NGF comprises at least the mutation NGF P61S or R100E.

The invention also provides method for the production of a mutant NGF comprising the steps of incubating the mutant pro-NGF as defined above with trypsin.

Preferably the incubation time is about 24 hours. Still preferably the incubation temperature is between about 4° to about 16° C. The weight ratio trypsin:pro-NGF may range from appr. 1:500 to appr. 1:5000, though the ratio 1:5000 is preferred, in particular when the incubation temperature is of about 16° C. and the incubation time is about 24 h and the proNGF mutant protein concentration is about 0.6 mg/ml. Such conditions work very well for the proNGF mutants and allow to set up a process time and cost saving.

The term “proNGF” or “pro-NGF” refers to the pro-form of human beta-NGF. The full human proNGF sequence is defined in SEQ ID NO: 1.

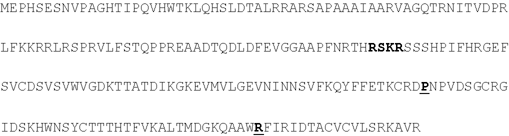

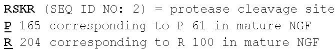

In order to obtain mature beta-NGF, the propeptide proNGF has to be cleaved by proteases. The prosequence of beta-NGF is a domain separate from the mature beta-NGF. Between these two domains, there is a native protease cleavage site Arg-Ser-Lys-Arg (referred herein to RSKR, SEQ ID NO: 2) at positions 101 to 104 of SEQ ID NO: 1. The cleavage site may be specifically processed by suitable proteases, in particular furin protease.

The term “proNGF mutant” or “proNGF mutein” refers to modifications of the pro-form of human beta-NGF by substitutions of amino acids. The proNGF mutein of the present invention is substituted at the native protease cleavage site RSKR (SEQ ID NO: 2) at least at both positions K and R corresponding to positions 101 and 103 of the human wild type proNGF sequence (SEQ ID NO: 1) by an amino acid selected from non-basic amino acids and Histidine.

In a preferred embodiment of the invention, amino acid Lysine in Position K (corresponding to position 103) is substituted with Alanine.

In another preferred embodiment of the invention, first amino acid Arginine in position R (corresponding to position 101) is substituted with Valine.

In another embodiment of the invention, the last amino acid arginine R corresponding to position 104 of the wildtype proNGF sequence (SEQ ID NO: 1) may also be substituted by any amino acid which allows processing of the proNGF by proteolytic cleavage to obtain beta NGF, preferably a basic amino acid such as Arginine or Lysine. For example, the presence of Alanine in Position R⁴ avoids processing of proNGF to beta NGF. Therefore, the mutant of invention cannot contain Alanine in position 104.

| SEQ | Protease cleavage site |
|---|---|
| ID NO | (pos. 101-104 of SEQ ID NO: 1) |
| 2 | RSKR (wild-type) |
| 3 | VSXR |
| 4 | XSXR |
| 5 | XSNR |
| 6 | VSAR |
| 7 | RXKR |
| 8 | XXXR |
| 9 | VXAR |

The term “non-basic amino acid” refers to any amino acid which is not positively charged. The term refers to an amino acid residue other than a basic amino acid. The term excludes amino acids Lysine or Arginine which are amino acids with positive side chains. Non-basic amino acids are negatively charged amino acids Glutamic Acid and Aspartic Acid, amino acids with polar uncharged side chains (Serine, Threonine, Asparagine, Glutamine), amino acids with hydrophobic side chains (Alanine, Valine, Isoleucine, Leucine, Methionine, Phenylalanine, Tyrosine, Tryptophane) and amino acids Cysteine, Glycine and Proline.

The term “biologically active pro-NGF” or “proNGF with biologically active conformation” as such refers to the biological activity of pro-NGF. A biologically active conformation of proNGF is determined by the presence of disulfide bridges occurring in natural beta-NGF. The activity may be, for example, determined according to an assay as described by Chevalier et al. 1994, Blood 83: 1479-1485, 1994, which is incorporated herein by reference. Example 11 describes an assay for the biological activity of proNGF via stimulation of the proliferation of TF1 cells. The term “beta-NGF” refers to a mature beta-nerve growth factor, preferably from human. The term “activity of beta-NGF” or “biologically active beta-NGF” as such means the biological activity of beta-NGF. Biologically active beta-NGF exists in the form of a dimer. Beta-NGF must be present in a dimeric form to have a biologically active conformation. The prerequisite of a biologically active conformation of beta-NGF is the correct formation of the disulfide bridges to a cystine knot. The activity may be, for example, determined according to the DRG assay (dorsal root ganglion assay), see for example Levi-Montalcini, R. et al., Cancer Res. 14 (1954) 49, and Varon, S. et al., Meth. in Neurochemistry 3 (1972) 203. In this assay the stimulation and survival of sensory neurons from dissociated dorsal root ganglia of chick embryos is monitored by means of neurite formation.

The term “substitution” or “substitutions” refers to modifications of the pro-form of human beta-NGF by replacement of amino acids. The term comprises the chemical modification of amino acids by e.g. substituting or adding chemical groups or residues to the original amino acid. The step of modification of the selected amino acids is performed preferably by site-specific mutagenesis. Preferably, the modification of proNGF is carried out by means of methods of genetic engineering for the mutation of the cDNA coding for proNGF. The modifications are mutations that cause the replacement of a single base nucleotide with another nucleotide of the genetic material. Point mutations results in encoding different amino acids compared to the wild-type sequence. Preferably, expression of the modified proNGF protein is then carried out in prokaryotic or eukaryotic organisms, most preferably in prokaryotic organisms.

The term “denaturating” or “denaturation” refers to a process in which the folding structure of a protein is altered. The term refers to unfold the tertiary structure of proNGF or proNGF mutein. The alteration of the folding structure is due to exposure to certain chemical or physical factors. As a result, some of the original properties of the protein, especially its biological activity, are diminished or eliminated. Due to the denaturing process, proteins become biologically inactive. Further, denatured proteins can exhibit a wide range of characteristics, including loss of biological function, loss of solubility and/or aggregation.

The term “refolding” or “renaturating” or “renaturation” refers to a process by which the protein structure assumes its native functional fold or conformation. Due to renaturation or refolding processes, the protein becomes biologically active.

The term “recombinant” refers to the cloning of DNA into vectors for the expression of the protein encoded by the DNA in a suitable host. The host is preferably a prokaryote, most preferably a bacterium. A “recombinant expression” as used herein refers to expression of proNGF or the proNGF mutein in in prokaryotic host cells, for example E. coli strains suitable for expression of recombinant proteins could be used.

The term “soluble” refers to a protein which is susceptible of being dissolved in some solvent. The term “insoluble” refers to a protein which is not susceptible of being dissolved in some solvent.

### BRIEF DESCRIPTION OF THE DRAWINGS

The present invention will be described by means of non-limiting examples in reference to the following figures

FIG. 1. HPLC Analysis of the purified proteolytic cleavage product from hproNGF P61SR100E. HPLC Analysis of the purified proteolytic cleavage product from hproNGF P61SR100E. The HPLC was carried out by using a reverse phase column (Jupiter® 5 μm C4 300 Å, LC Column 250×4.6 mm). The buffers were: A) 0.05% TFA in Water B) 0.05% TFA in CH3CN. The elution profile showed the presence of a main peak (2) and two secondary peaks (1,2). The fractions corresponding to the three peaks were run on a MALDI TOF mass spectrometer (not shown), giving the following results: Peak 1=13411 Da, Peak 2=13254 Da, Peak 3=12205 Da. The three identified molecular species are compatible with three different NGF form, precisely: peak 1 with mature NGF plus the N-terminus Arginine, peak2 with the expected mature NGF form, peak 3 with the desocta NGF form (Holland, D. R. (1994). J. Mol. Biol).

FIG. 2A. Plasmid Pet 11a map

FIG. 2B. Illustrates the pET-11a-d cloning expression region
(1) SEQ ID NO: 12;
(2) SEQ ID NO: 13;

(3) SEQ ID NO: 14;
(4) SEQ ID NO: 15 (Pet-11b nucleic acid);
(5) SEQ ID NO: 16 (Pet-11b peptide);
(6) SEQ ID NO: 17 (Pet-11c.d nucleic acid);
(7) SEQ ID NO: 18 (Pet-11c.d peptide);
(8) SEQ ID NO: 19 (Pet-11d nucleic acid); and
(9) SEQ ID NO: 20.

FIG. 3A. hproNGF wt purification: example of ion-exchange chromatography and

FIG. 3B hydrophobic-interaction chromatography.

FIG. 4A. HproNGF P61SR100E purification: example of ion-exchange chromatography, and

FIG. 4B hydrophobic-interaction chromatography.

FIG. 5. hNGF wt purification: example of ion-exchange chromatography.

FIG. 6. SDS-PAGE of the fractions corresponding to the peak eluted from ion-exchange chromatography of hNGF wt.

FIG. 7. hNGF P61SR100E purification: example of ion-exchange chromatography.

FIG. 8A. hproNGF wt VSAR purification: example of ion-exchange chromatography and

FIG. 8B. hydrophobic-interaction chromatography.

FIG. 9A. hproNGF P61SR100E VSAR purification: example of ion-exchange chromatography, and

FIG. 9B. hydrophobic-interaction chromatography.

FIG. 10A. Proteolytic cleavage tests in small scale (hproNGF wt VSAR). Conditions of proteolytic cleavage: 4° C., overnight (o/n), trypsin:protein ratio of 1:250, 1:500, 1:1000.

FIG. 10B. Proteolytic cleavage tests in small scale (hproNGF wt VSAR). Conditions of proteolytic cleavage: 22° C., overnight, trypsin:protein ratio of 1:250, 1:500, 1:1000.

FIG. 10C. Proteolytic cleavage tests in small scale (hproNGF wt VSAR). Conditions of proteolytic cleavage: 16° C., overnight, trypsin:protein ratio of 1:1000, 1:5000, 1:10000.

FIG. 11A. Proteolytic cleavage tests in large scale (hproNGF wt VSAR) Ion-exchange chromatography (HiTrap SP XL, 5 ml) of the proteolytic product of 3 mg of hproNGF wt, and

FIG. 11B. hproNGF wt VSAR incubated at 22° C. for 24 hours with trypsin at a trypsin:protein ratio of 1:500.

FIG. 12A. Proteolytic cleavage tests in large scale (hproNGF wt VSAR). Ion-exchange chromatography (HiTrap SP XL, 5 ml) of the proteolytic cleavage product of 3 mg of hproNGF wt, and

FIG. 12B. hproNGF wt VSAR incubated at 30° C. for 2 hours with trypsin at a trypsin:protein ratio of 1:500.

FIG. 13. Proteolytic cleavage tests in large scale (hproNGF wt VSAR). SDS-PAGE of the proteolytic product of 3 mg of hproNGF wt and wt VSAR incubated at 22° C. for 24 hours with trypsin at a trypsin:protein ratio of 1:500 and at 30° C. for 2 hours with trypsin at a trypsin:protein ratio of 1:500.

FIG. 14A. Proteolytic cleavage tests in large scale (hproNGF wt VSAR). Ion-exchange chromatography (HiTrap SP XL, 5 ml) of the proteolytic product of 3 mg of hproNGF wt, and

FIG. 14B. hproNGF wt VSAR incubated at 25° C. for 24 hours with trypsin at a trypsin:protein ratio of 1:500.

FIG. 15A. Proteolytic cleavage tests in large scale (hproNGF wt VSAR). Ion-exchange chromatography (HiTrap SP XL, 5 ml) of the proteolytic product of 3 mg of hproNGF wt, and

FIG. 15B. wt VSAR incubated at 37° C. for 1 hour with trypsin at a trypsin:protein ratio of 1:500.

FIG. 16A. Proteolytic cleavage tests in large scale (hproNGF wt VSAR). Ion-exchange chromatography (HiTrap SP XL, 5 ml) of the proteolytic product of 3 mg of hproNGF wt and

FIG. 16B wt VSAR incubated at 22° C. for 24 hours with trypsin at a trypsin:protein ratio of 1:500, pH 8.

FIG. 17A. Proteolytic cleavage tests in large scale (hproNGF wt VSAR). Ion-exchange chromatography (HiTrap DEAE FF 5 ml) of the proteolytic product of 3 mg of hproNGF wt, and

FIG. 17B. wt VSAR (right) incubated at 22° C. for 24 hours with trypsin at a trypsin:protein ratio of 1:500, pH 8.

FIG. 18. SDS-PAGE of hNGF WT cleaved from hproNGF WT VSAR by using different proteolytic protocols. Three representative SDS-PAGE of the proteolytic cleavage products from hproNGF WT VSAR (compared side by side to hproNGF WT). Different cleavage protocols were analyzed. Legend: (A) Lanes 1) Molecular Weight standard; 2) hproNGF wt and hNGF wt (controls) 3) Purified hNGF wt (on DEAE column). Protocol: 22° C. 24 h trypsin:protein=1:500 4) Purified hNGF wt VSAR (on DEAE column). Protocol: 22° C. 24 h tripsin:protein=1:500 5) Purified hNGF wt (SP Sepharose column) protocol: 22° C. 24 h trypsin:protein=1:500 6) Purified hNGF wt VSAR (SP Sepharose column) protocol: 22° C. 24 h trypsin:protein=1:500. (B) Lanes 1) Molecular Weight standard; 2) hproNGF wt and hNGF wt (controls) 3) Purified hNGF wt (SP Sepharose column) protocol: 25° C. 24 h trypsin:protein=1:500 6) Purified hNGF wt VSAR (SP Sepharose column) protocol: 25° C. 24 h trypsin:protein=1:500. (C) Lanes 1) Molecular Weight standard; 2) hproNGF wt and hNGF wt (controls) 3) Purified hNGF wt (SP Sepharose column) Protocol: 37° C. 1 h trypsin:protein=1:500 4) Purified hNGF wt (SP Sepharose column) Protocol: 37° C. 1 h trypsin:protein=1:500.

FIG. 19 A. Mass spectrum of band of 25 kDa obtained by proteolysis of the control.

FIG. 19 B. Mass spectrum of band of 25 kDa obtained by proteolysis of hproNGF wt.

FIG. 19 C. Mass spectrum of band of 25 kDa obtained by proteolysis of hproNGF wt VSAR

FIG. 20. Western Blot Analysis on NGF WT VSAR derived from the proteolytic cleavage of hproNGF WT and hproNGF WT VSAR. The purified hNGF (4 μg) derived from the proteolytic cleavage of hproNGF WT and hproNGF WT VSAR were analyzed by Western Blot. The membrane was challenged with anti NGF (H-20 Santa Cruz). Legend: Lanes 1) Purified hNGF wt. Protocol: 25° C. 24 h trypsin:protein=1:500; 2) Purified hNGF wt VSAR. Protocol: 25° C. 24 h trypsin:protein=1:500; 3) Purified hNGF wt. Protocol: 22° C. 24 h trypsin:protein=1:500; 4) Purified hNGF wt VSAR. Protocol: 22° C. 24 h trypsin:protein=1:500.

FIG. 21 A. Proteolytic cleavage tests in small scale (hproNGF P61SR100E VSAR) Conditions of proteolytic cleavage: 4° C., incubation times: 90 minutes, 2 hours, 4 hours, overnight, trypsin:protein ratio of 1:250.

FIG. 21 B. Proteolytic cleavage tests in small scale (hproNGF P61SR100E VSAR) Conditions of proteolytic cleavage: 4° C., incubation times: 90 minutes, 2 hours, 4 hours, overnight, trypsin:protein ratio of 1:1000.

FIG. 21 C. Proteolytic cleavage tests in small scale (hproNGF P61SR100E VSAR) Conditions of proteolytic cleavage: 4° C., incubation times: 90 minutes, 2 hours, 4 hours, overnight, trypsin:protein ratio of 1:250.

FIG. 21 D. Proteolytic cleavage tests in small scale (hproNGF P61SR100E VSAR) Conditions of proteolytic cleavage: temperature: 4° C., 20° C., 30° C.; incubation times: 60 minutes, 4 hours, overnight, 24 hours; trypsin:protein ratio of 1:5000, 1:10000.

FIG. 21 E. Proteolytic cleavage tests in small scale (hproNGF P61SR100E VSAR) Conditions of proteolytic cleavage: temperature: 16° C., 20° C., 30° C.; incubation times: 60 minutes, 4 hours, overnight; trypsin:protein ratio of 1:5000, 1:10000.

FIG. 21 F. Proteolytic cleavage tests in small scale (hproNGF P61SR100E VSAR) Conditions of proteolytic cleavage: temperature: 4° C., 16° C.; incubation times: overnight, 24 hours; trypsin:protein ratio of 1:1000, 1:5000.

FIG. 22A. Proteolytic cleavage tests in large scale (hproNGF P61SR100E VSAR). Ion-exchange chromatography (HiTrap SP XL, 5 ml) of the proteolytic product of 3 mg of hproNGF P61SR100E VSAR incubated at 4° C. o/n at a trypsin:protein ratio of 1:1000, and

FIG. 22B. incubated at 16° C. for 24 hours with trypsin at a trypsin:protein ratio of 1:5000.

FIG. 23. Proteolytic cleavage tests in large scale (hproNGF P61SR100E VSAR). SDS-PAGE of the proteolytic product of 3 mg of hproNGF P61SR100E VSAR incubated at 16° C. for 24 hours with trypsin at a trypsin:protein ratio of 1:5000 and at 16° C. for 24 hours with trypsin at a trypsin:protein ratio of 1:5000.

FIG. 24A. Proteolytic cleavage tests in large scale (hproNGF P61SR100E VSAR). Ion-exchange chromatography (HiTrap SP XL, 5 ml) of the proteolytic product of 13 mg of hproNGF P61SR100E VSAR incubated at 4° C. o/n at a trypsin:protein ratio of 1:1000, and

FIG. 24B. at 16° C. for 24 hours with trypsin at a trypsin:protein ratio of 1:5000.

FIG. 25. SDS-PAGE of hNGF P61SR100E cleaved from hproNGF P61SR100E VSAR by using two different cleavage protocols. A representative SDS-PAGE of hNGF P61SR100E cleaved from hproNGF P61SR100E VSAR by the two protocol conditions listed in the legend above. Each lane was loaded with 10 μg of the digestion product. Legend: Lane 1: hNGFP61SR100E VSAR, cleavage protocol: 4° C. 24 h trypsin:protein=1:1000; Lane 2: hNGFP61SR100E VSAR, cleavage protocol: 16° C. 24 h tripsin:protein=1:5000; Lane 3: Molecular Weight Marker.

FIG. 26. Mass Spectrometry Analysis on hNGF P61SR100E. hNGF P61SR100E, obtained from the purified proteolytical cleavage of hproNGF P61SR100E VSAR (at 16° C. for 24 h with a tripsin:protein ratio of 1:5000), was analyzed by Mass Spectrometry (Maldi TOF).

FIG. 27. Cell proliferation assay TF1 of hNGF P61SR100E obtained from proteolytic cleavage of hproNGF P61SR100E VSAR.

FIG. 28. Cell proliferation assay TF1 of hNGF wt obtained from proteolytic cleavage of hproNGF wt VSAR.

FIG. 29. Maldi TOF Mass Spectrometry analysis of non-purified hproNGF P61SR100E, hproNGF P61S VSAR and hproNGF R100E VSAR cleavage products. MALDI TOF Mass Spectrometry analysis. 120 μg of hproNGF P61SR100E, hproNGF P61S VSAR and hproNGF R100E VSAR were incubated with trypsin by using two different protocols, and then analyzed directly by Mass Spectrometry. Protocol 1: 16° C. 24 h trypsin:protein=1:5000 (Panel A). Protocol 2: 4° C. 24 h trypsin:protein=1:1000 (Panel B).

FIG. 30. Maldi TOF Mass Spectrometry analysis of purified hproNGF P61SR100E, hproNGF P61S VSAR and hproNGF R100E VSAR cleavage products. MALDI TOF Mass Spectrometry analysis. 3 mg of hproNGF P61SR100E, hproNGF P61S VSAR and hproNGF R100E VSAR were incubated with trypsin at 16° C. for 24 h with a trypsin:protein ratio: 1:5000, purified, and then analyzed by Mass Spectrometry.

FIG. 31. Maldi TOF Mass Spectrometry analysis of purified hproNGF P61SR100E, hproNGF P61S VSAR and hproNGF R100E VSAR cleavage products. MALDI TOF Mass Spectrometry analysis. 3 mg of hproNGF P61SR100E, hproNGF P61S VSAR and hproNGF R100E VSAR were incubated with trypsin by using three different protocols, purified, and then analyzed by Mass Spectrometry. Protocol 1: 16° C. 17 h trypsin:protein=1:5000 (black). Protocol 2: 16° C. 4 h trypsin:protein=1:5000 (red). Protocol 3: RT 4 h trypsin:protein=1:5000 (tested only for hproNGF P61SR100E VSAR).

FIG. 32. SDS-PAGE of purified proteolytic products from hproNGF P61SR100E VSAR and hproNGF WT VSAR. A representative SDS-PAGE of purified proteolytic products from hproNGF P61SR100E VSAR and hproNGF WT VSAR (3 mg), digested in the two conditions indicated in the legend above. Each lane was loaded with 10 μg of the digestion product. Legend: Lanes 1) Purified hNGF wt from hproNGF WT VSAR. Protocol: Room Temperature, 18 h, trypsin:protein=1:10000; 2) Purified hNGF P61SR100E from hproNGF P61SR100E VSAR. Protocol: Room Temperature, 18 h, trypsin:protein=1:10000; 3) Purified hNGF wt from hproNGF WT VSAR. Protocol: 16° C., 24 h, trypsin:protein=1:5000; 4) Purified hNGF P61SR100E from hproNGF P61SR100E VSAR. Protocol: 16° C., 24 h, trypsin:protein=1:5000.

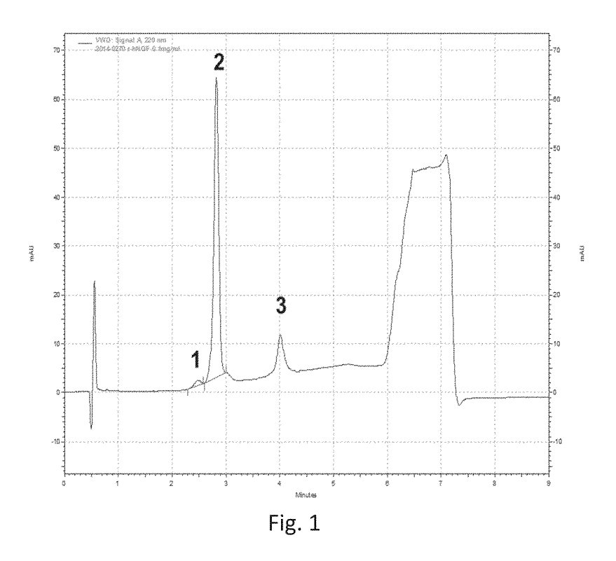

### DETAILED DESCRIPTION OF THE INVENTION

### Materials and Methods

### Expression and Purification of Human NGF WT and Human Mutant NGF P61SR100E

Growth and Inducing of Production of Human proNGF in E. Coli Cells

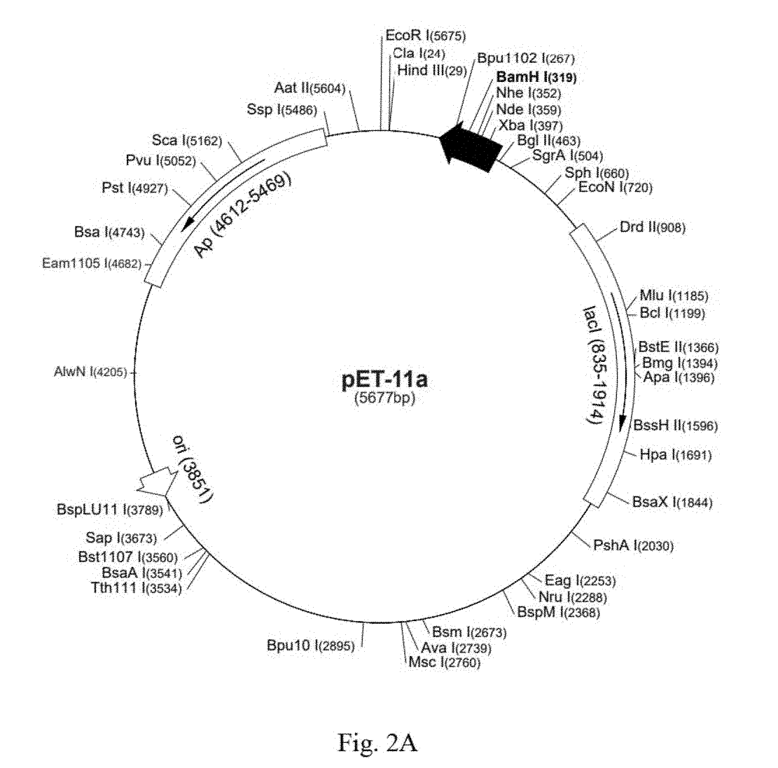

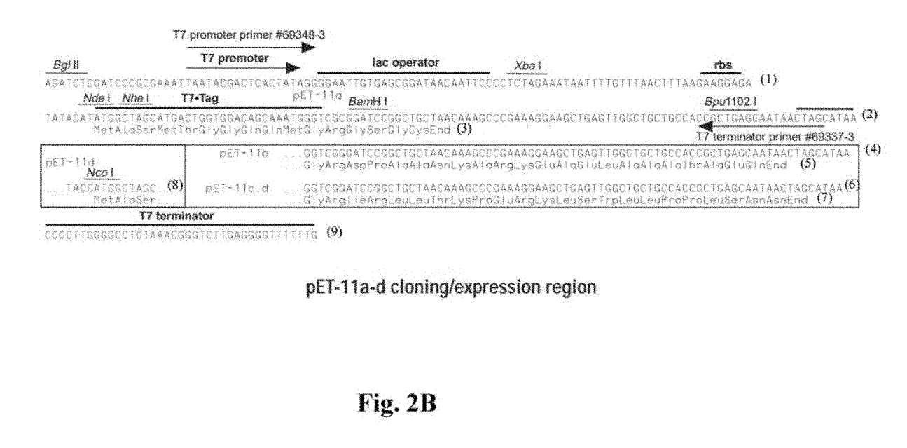

Two flasks, each containing 100 ml of medium LB supplemented with ampicillin, (LB: tryptone (Fluka) 10 g/l; yeast extract (BD) 5 g/l; NaCl (AppliChem) 5 g/l, ampicillin (Applichem) 1 mM) are inoculated from glycerol stocks of Rosetta E. Coli cells (DE3) (PLysS) already transformed with plasmid Pet11a (Novagen) (FIG. 2), with the sequence of DNA coding respectively for human proNGF wild type (hproNGF wt) and human proNGF P61SR100E (hproNGF P61SR100E).

The cultures are incubated at 37° C. overnight under stirring (200 rpm) in an incubator INFORS (HT). The presence of the antibiotic avoids contaminations, enabling the growth of cells containing the plasmid Pet11a that, by carrying β-lactamase enzyme gene, confers resistance to the ampicillin. Four flasks containing 850 ml of medium LB, with ampicillin (AppliChem) 1 mM are inoculated with 17 ml/flask of pre-inoculum (pre-inoculum:inoculum ratio of 1:50). The cultures are left to grow at 37° C. under stirring for about 3 hours, or until the optical density, measured under a spectrophotometer at a wavelength of 600 nm, reaches a value between 1.0 and 1.2. The expression of the coding gene is then induced for the proNGF by adding isopropyl β-D-1-thiogalactopyranoside (IPTG) (AppliChem) 1 mM. After induction, the cultures are incubated at 37° C. for another 4 hours under stirring. The bacterial broth is centrifuged at 8000 rpm for 20 minutes at 4° C. (Beckman, JA-10 rotor), in order to separate the cells from the culture medium. The pellet obtained is weighed and frozen at −20° C.

### Lysis to Obtain Inclusion Bodies

The proNGF expressed by cells is accumulated in inclusion bodies (Paoletti et al. 2009). The cells are lysed to then isolate the inclusion bodies. The bacterial pellet, stored at −20° C., is thawed and then re-suspended in a Tris HCl 0.1 M buffer and ethylenediaminetetraacetic acid or EDTA (AppliChem) 1 mM pH 7.0 (5 ml/g pellet), with an Ultra-Turrax T25 disperser (IKA). After adding 1.5 mg/g of lysozyme (AppliChem), 10 μg/ml of DNase (Sigma) and magnesium chloride (MgCl₂, Merck) 3 mM, the bacterial lysate is incubated for 90 minutes on the magnetic stirrer (200 rpm) at room temperature. The lysate is then fluidised through three cycles of sonication at full intensity, lasting 60 seconds each, interspersed with an incubation period of 60 seconds in ice (sonicator VibraCell, Sonics).

### Preparation of the Inclusion Bodies

The cell lysate is supplemented with a triton buffer (50% of the volume, 6% triton X-100 (AppliChem), NaCl (AppliChem) 1.5 M, EDTA (AppliChem) 60 mM), incubated for 30 minutes under stirring (200 rpm) and then centrifuged at 12000 rpm at 4° C. for 30 minutes (Beckman, JA-30.50 rotor). The pellet is re-suspended in an appropriate volume of a solution of Tris HCl 0.1 M and EDTA (AppliChem) 1 mM, pH 7 with an Ultra-Turrax disperser. The step of re-suspension in the triton buffer, incubation and centrifugation is repeated twice. The inclusion bodies thus obtained are subjected to washing with buffer TrisHCl 0.1 M, EDTA (Applichem) 20 mM, pH 7.0. The second centrifugation pellet is re-suspended with Ultra-Turrax in an appropriate volume of the buffer TrisHCl 0.1 M, EDTA (AppliChem) 20 mM, pH 7.0 and centrifuged again at 12000 rpm at 4° C. for 20 minutes (Beckman, JA-30.50 rotor). The wash is repeated 3-4 times. The inclusion bodies are weighed and frozen at −20° C.

### Solubilisation of Inclusion Bodies

The inclusion bodies are thawed and solubilised with 5 ml/g of guanidine chloride solution HCl 6M (AppliChem), in a buffer 0.1 M Tris HCl pH 8.00, 1 mM EDTA (AppliChem) to which the reducing agent dithiothreitol (DTT, AppliChem) 100 mM is added fresh. After incubation for 2-3 hours on the wheel, the inclusion bodies are completely solubilised. The solution is brought to a pH of about 3.00-4.00 with HCl (Sigma) 5 M and centrifuged at 12000 rpm for 20 minutes (Beckman, JA-30.50 rotor). The supernatant was dialysed against guanidine chloride (AppliChem) 6 M pH 3.00-4.00 at 4° C. to eliminate the DTT. Lowering the pH avoids the formation of random intermolecular disulfide bridges. The reducing environment obtained with excess of protons, in fact, compensates elimination by dialysis of the reducing agent DTT. The dialysis is changed 4 times (2 dialysis over-day and 2 overnight).

The total proteins present in the dialysed solution are quantified by Bradford's method, based on the use of a commercial reagent consisting of Brilliant Blue G (Coomassie blue) in phosphoric acid and methanol. The dye binds the proteins by electrostatic bonds with its sulfonic groups. Coomassie Blue has a peak absorbance at 475 nm, which shifts to 595 nm when it bonds the proteins, developing a blue colouration. The intensity of the colouration determined at 595 nm is directly proportional to the protein concentration in the solution. The concentration of the protein of interest is obtained by means of a calibration curve, constructed by making the Bradford reagent react with increasing quantities of a solution of Bovine Serum Albumin (BSA) with known concentration: 0, 0.2, 0.4, 0.6, 0.8, 1 μg/μl in guanidine chloride (AppliChem) 3 M.

### Refolding

The refolding protocol by the method of pulses has been optimised for the murine proNGF (Paoletti et al. 2009). To obtain the correct protein refolding, 50 μg/ml of protein are added to the refolding buffer at 60-minute intervals: Tris HCl 0.1 M, L-Arginine (AppliChem) 1 M, EDTA (AppliChem) 5 mM, 0.61 gr/L oxidised glutathione (AppliChem) and 1.53 gr/l reduced glutathione (AppliChem) pH 9.5 at 4° C. The volume of the refolding buffer used must be such as to dilute the guanidine chloride until the concentration of 200 mM.

$$VOLUME OF REFOLDING BUFFER * \frac{\begin{matrix}INITIAL VOLUME OF INCLUSION BODIES \times \\ [ GUANIDINE CHLORIDE ] ( 6 M )\end{matrix} }{FINAL [ GUANIDINE CHLORIDE ] ( 200 mM )}$$

Human proNGF Purification: Ion-Exchange Chromatography

The refolding solution is concentrated up to a volume equal to about a third of the initial volume and then dialysed at 4° C. for about 18 hours against a volume of sodium phosphate buffer 50 mM pH 7.0 100 times greater than the volume of the sample. After dialysis, the sample is centrifuged at 10000 rpm for 30 minutes at 4° C. (Beckman, JA-10 rotor). The supernatant is recovered and filtered, and then purified by ion-exchange chromatography. Ion-exchange chromatography is a method of separation of mixtures of analytes based on an ion-exchange balance between a resin (stationary phase) and a mobile electrolyte solution. The stationary phase consists of macromolecules containing electrically charged functional groups that interact with oppositely charged groups present in the mobile phase that passes through it. The sample to be purified must be in a buffer with a pH and ionic strength such as to ensure the bonding to the resin of analytes that you want to separate. The resin used is HiLoad 26/10 SP Sepharose High Performance (GE Healthcare). It is a strong cation exchange resin consisting of an agarose matrix to which the negatively charged sulfonated groups are bonded. The hproNGF wt proteins and P61SR100E are in a sodium phosphate buffer 50 mM, pH 7.0. Exposed at a pH lower than its isoelectric point (pI) (pI of hproNGF wt: 9.89, pI of hproNGF P61SR100E: 9.69), the proNGF is positively charged and is therefore attracted by the cation exchange resin. The column is connected to the apparatus for automated purification ÄKTA pure (GE Healthcare) and the resin is balanced according to the instructions through the passage of: 5 column volumes of buffer A (sodium phosphate 50 mM, pH 7.0), 5 column volumes of buffer B (sodium phosphate 50 mM pH 7.0, 1M NaCl (Applichem)) followed by 5 column volumes of buffer A. The sample is loaded onto the column with the automated system System Loading. After loading, the resin is washed with two column volumes of buffer A. Elution occurred through the formation of a gradient from 0% to 100% of buffer B in 6 column volumes. Ionic competition by NaCl allows the release of the protein from the resin. Each protein eluted exiting the chromatographic column passes through a detector that records a signal. A chromatogram is obtained at the end of each chromatography: a graph that shows the variation of the signal recorded by the detector at the passage of the analytes as a function of time (or, in our case, the volume of eluent), starting from the moment when the mixture is introduced in the column. At the passage of the eluent alone, the signal is very low and is represented on the graph as a baseline. At the passage of the proteins under examination, the signal begins to increase until it reaches a maximum peak and then returns to the baseline. At the end of the chromatography, the inventors obtain peak-shaped profiles that correspond to the different molecules present in the mixture. The position of the peaks on the time axis (or the volume of eluent) depends on the time (or volume) of retention, namely on the retention time of the molecule within the column. A compound that does not interact with the stationary phase travels at the same speed of the mobile phase, while a compound that interacts with the stationary phase is instead delayed. Different substances can be separated if they have different retention times. The retention time, given the same experimental conditions, is characteristic for each protein. The highest peak represents the moment when the highest fraction of molecules of that substance passes through the detector. The area under the peaks is proportional to the quantity of each component. The fractions corresponding to the peak of elution of hproNGF are merged, brought to a final concentration of 1 M with ammonium sulfate (AppliChem) and stored at 4° C.

Human proNGF Purification: Hydrophobic Interaction Chromatography

The sample is centrifuged at 12000 rpm for 20 minutes at 4° C. (Beckman, JA-30.50) to precipitate insoluble proteins to a concentration of ammonium sulfate 1 M. The supernatant is recovered and filtered. The sample is purified by hydrophobic interaction chromatography to separate the proteins based on their surface hydrophobicity. The ammonium sulfate, by removing the water molecules that shield the hydrophobic regions of the proteins in solution, allows the exposure of hydrophobic protein regions that can interact with a stationary phase consisting of hydrophobic groups. The column used is HiLoad 26/10 Phenyl Sepharose High Performance (GE Healthcare). The resin consists of an array of high cross-linking agarose to which the phenyl groups are bonded and which give the resin a high degree of hydrophobicity. The column is connected to the apparatus for automated purification ÄKTA pure (GE Healthcare) and is balanced according to the instructions: 2 column volumes of buffer A (sodium phosphate 50 mM, pH 7.0) followed by 10 column volumes of buffer B (sodium phosphate 50 mM, pH 7.0, 1 M ammonium sulfate (Applichem)). At this point, the sample is loaded. After loading, the resin is washed with two column volumes of buffer A. Elution occurred through the formation of a linear gradient of decreasing ionic strength from 100% to 0% of ammonium sulphate in 6 column volumes using buffer A. The fractions corresponding to the peak of elution of hproNGF are merged and dialysed for about 18 hours at 4° C. against an appropriate volume of buffer A. HproNGF and P61SR100E are quantified by UV-visible absorbance spectroscopy, using the UV-visible spectrophotometer NanoDrop, which makes it possible to measure micro-volumes of sample up to 1 μl. From the value of absorbance at 280 nm, it is possible to measure their protein concentration in mg/ml by applying Lambert-Beer's law, knowing the molecular weight of the monomer of hproNGF wt: 24847.2 Da and hpro NGF P61SR100E: 24810.1 Da and the molar extinction coefficient (E), which is the same for both proteins and equal to 25355. The proteins obtained are either aliquoted out and stored at −20° C. or subjected to proteolytic cleavage to yield the corresponding mature protein.

Proteolytic Cleavage of Human proNGF with Trypsin

The protocol of proteolytic cleavage with trypsin is modified from that described for murine proNGF (Paoletti et al. 2009) and foresees incubation in a thermostated bath under the following conditions:
for 18 hours at 16° C. with trypsin at a ratio of trypsin:hproNGF wt of 1:150 for hproNGF wt
for 1 hour at 30° C. with trypsin at a ratio of trypsin:hproNGF P61SR100E of 1:250 for hproNGF P61SR100E.

### Human NGF Purification: Ion-Exchange Chromatography

The product of proteolysis is purified by ion-exchange chromatography. The resin used is SP Sepharose (strong cation exchange) consisting of long chains of dextran linked to an agarose matrix with high cross-linking degree (HiTrap SP XL, 5 ml). The functional group of the resin is a sulfonate group that provides the negative charge needed to electrostatically attract the cations on the basis of their opposite charge. NGF exposed to a lower pH of its pI (hNGF wt: 8.81; hNGF P61SR100E: 8.24) is positively charged and is attracted by the cation exchange resin. The column is connected to the apparatus for automated purification ÄKTA pure (GE Healthcare) and purification takes place as described above for the resin HiLoad 26/10 SP Sepharose High Performance. The fractions eluted corresponding to hNGF wt and P61SR100E are merged and concentrated by centrifugation at 3000 g, 4° C. (Beckman, Allegra 25R) in a concentrator Vivaspin 15R (Sartorius), disposable ultra-filtration device for oscillating centrifuges used for the concentration of biological samples of volumes between 2 and 15 ml. HNGF wt and P61SR100E are then quantified using the UV-visible spectrophotometer NanoDrop, knowing the molecular weight of the monomer (hNGF wt: 13267.1 Da; hNGF P61SR100E: 13230 Da) and the molar extinction coefficient, which is the same for both proteins and equal to: 19855.

### Polyacrylamide Gel Electrophoresis Under Denaturing Conditions

In order to make sure that the proteolytic cleavage has taken place efficiently, i.e. that the whole proNGF (wt and P61SR100E) has been proteolyzed in NGF, the peak fractions of the chromatography are subjected to polyacrylamide gel electrophoresis under denaturing conditions (SDS-PAGE: Sodium Dodecyl Sulphate-PolyAcrylamide Gel Electrophoresis). The gel is prepared with a concentration of acrylamide of 15%. The running gel (the bottom part of the gel in which the separation of proteins based on the molecular weight will take place) is prepared first and consists of: double-distilled H₂O (dd), Tris HCl pH 8.8, Sodium Dodecyl Sulphate (SDS) (Applichem) at 10% and acrylamide (nzytech). The catalysts for the radical polymerisation of acrylamide-ammonium persulphate (APS) and Tetramethylethylenediamine (TEMED, Sigma)—are added to the running gel. Subsequently, the upper stacking gel is prepared, in which the wells are housed and that makes it possible for the proteins to arrive at the same time at the front of the running gel, consisting of: ddH₂O, Tris HCl pH 6.8, SDS (Applichem) at 10% and acrylamide (nzytech), to which are added the catalysts APS and TEMED (Sigma). The gel obtained after polymerisation and contained between the two specific slides is transferred into the electrophoretic chamber and capped with a running buffer consisting of: glycine (Sigma) 192 mM, Tris Base (Sigma) 25 mM, SDS (Applichem) at 0.1%, H₂O. The protein samples to be analysed on the gel are prepared by adding a loading buffer (buffer Dye 4× consisting of: Tris HCl 0.5 M pH 6.8, glycerol (Applichem) at 10%, SDS (Applichem) at 10% and bromophenol blue 1%) and DTT (Applichem), so as to have a final concentration of 0.1 M in a total volume of 20 μl. The samples are boiled for 10 minutes and then loaded on the gel along with a molecular weight marker: Prestained Protein Marker VI (AppliChem), covering a range of molecular weights between 10 and 245 kDa. The chamber for the electrophoretic run is connected to a power unit set to 40 mA. After the electrophoretic run, the gel is treated with a commercial dye: EzWay™ Protein-Quick Blue Staining Solution (KOMABIOTECH) in order to detect the bands corresponding to proteins.

### Site-Directed Mutagenesis: Production of Mutants VSAR

Extraction and Sequencing of Plasmids Pet11a Containing Coding Sequences of Human proNGF (Wild-Type) and Human proNGF P61SR100E

The plasmids pet11a-hproNGF wt and pet11a-hproNGF P61SR100E that will undergo site-directed mutagenesis are first checked by sequencing the coding portion. The plasmids to be sent to the sequencing service are extracted from the cells of E. Coli by mini-preparation of plasmid DNA using a QUIAGEN Plasmid Mini kit, following the instructions of the manual. From stock in glycerol (10%), the E. Coli cells (Rosetta (DE3)), transformed with plasmids Pet 11a-hproNGF wt and Pet 11a-hproNGFP61SR100E are inoculated in 5 ml of sterile medium LB. The inocula are incubated at 37° C. for about 18 hours under stirring at 200 rpm. The bacterial growths are centrifuged at 6000 g for 15 minutes at 4° C. The pellet is re-suspended in 0.3 ml of buffer P1, consisting of: Tris-HCl 50 mM, EDTA 10 mM, pH 8.0 to which a solution of RNase A has been added previously at a concentration of 100 ug/ml. 0.3 ml of buffer 2 are added consisting of: 1% SDS, an ionic detergent that destroys the cell membranes; and 0.2 M NaOH, which denatures the macromolecules by changing the state of ionisation of the groups that form them. After mixing it well, the solution is incubated at 15° C.−25° C. for 5 minutes. After this step of alkaline cell lysis, 0.3 ml of buffer 3 are added to the solution, previously brought to a temperature of 4° C. and consisting of potassium acetate, 3 M, pH 5.5, which restores the pH of the solution to neutral, allowing precipitation of SDS and the renaturation of the macromolecules. Unlike the other molecules, which are unable to regain their correct native conformation, but form non-specific bonds between them, generating a “multi-molecular” aggregate, the plasmid, being a small, super-wound circular molecule, does not denature completely and, in the process of renaturation, resumes its correct native form. The solution is incubated on ice for 5 minutes to then be centrifuged at 15000 g (centrifuge eppendorf 5415 R) for 10 minutes at 4° C. This causes all the (aggregated) macromolecules to precipitate. The supernatant containing DNA is recovered and transferred by gravity into a QIAGEN-Tip, previously balanced with 1 ml of buffer QBT consisting of: NaCl 750 mM, MOPS 50 mM, pH 7.0, isopropanol at 15%, triton X-1000 15%. Before proceeding with DNA elution with 0.8 ml of buffer QF consisting of NaCl 1.25 M, Tris-HCl 50 mM pH 8.5 and isopropanol at 15%, two washes are performed with 2 ml of buffer QC consisting of NaCl 1 M, MOPS 50 mM pH 7.0 and isopropanol at 15%. All the solutions pass through the resin by gravity. The eluted DNA is precipitated after adding 0.7 volumes of isopropanol and recovered after centrifugation at 15000 g for 30 minutes at 4° C. The pellet obtained is washed with 1 ml of 70% ethanol and centrifuged at 15000 g for 10 minutes. After discarding the supernatant, the pellet is made to dry out in air and re-dissolved in an appropriate volume of H₂O. The plasmids are quantified using UV-Visible absorption spectroscopy at Nanodrop and 900 ng are sent to the Genechron DNA sequencing service (www.genechron.com). The primers used for sequencing are T7 promoter and T7 terminator, whose sequence is described on the plasmid map (FIG. 2, SEQ ID NO: 12 and SEQ ID NO: 13, respectively).

The Val101Arg and Ala103Lys mutations (Table 0) are placed in the consensus sequence for the proteolytic cleavage by the furin endoprotease in hproNGF wt and hproNGF P61R100E via site-directed mutagenesis, using the commercial mutagenesis kit QuikChange II XL Site-Directed Mutagenesis manufactured by Agilent Technologies. The primers (two complementary oligonucleotides containing Val101Arg and Ala103Lys mutations) are drawn according to the instructions specified by the protocol: the desired mutation is sandwiched in the middle on both oligonucleotides, with a length ranging between 25 and 45 bases, and presents on both sides 10-15 bases of the correct sequence. The oligonucleotides have a content of at least 40% of pairs of guanidine/cytosine (GC) bases and end with one or more pairs of GC bases, so that the melting temperature, calculated using the following formula:

Tm=81.5+0.41(% GC)−(675/N)−% MISMATCH

(where N is the number of bases that form the primers) is less than or equal to 78° C. The two complementary oligonucleotides containing the desired mutation are synthesised by the service Invitrogen™ Custom DNA Oligos (Life Technologies). Once the inventors have obtained the oligonucleotides (Primer 1, sense: 12452.2 μg/μmoles and Primer 2, anti-sense: 12763.2 μg/umoles, Table 0), the inventors prepare the reactions for the PCR (Polymerase Chain Reaction) by adding two micro-tubes Eppendorf:
5 μl of 10× reaction buffer provided by the kit
1.25 μl of primer 1 (sense) previously diluted to obtain a solution with a final concentration of 1 μg/μl
1.25 μl of primer 2 (anti-sense) previously diluted to obtain a solution with a final concentration of 1 μg/μl
1 μl of dNTP mix provided by the kit
1.5 μl of Quick solution reagent provided by the kit

To this solution the inventors add 40 ng of plasmid. In each tube, a final volume of 50 μl is reached by adding ddH₂O. After having added 1 μl of enzyme Quick change supplied by the kit, the reactions are subjected to PCR according to the pattern shown in the table (Table 1).

TABLE 0
Forward and reverse primers used in site-directed mutagenesis
of hproNGF wt and hproNGF P61SR100E.
(Primer Sequence 5′ to 3′ SEQ ID NO: 10;
Complement 5′ to 3′ SEQ ID NO: 11).

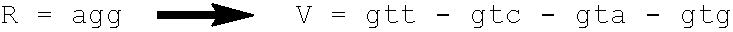

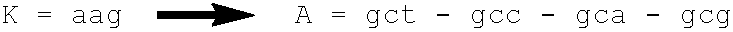

Tm = 79.3 degrees C., Bases = 41,
GC content = 58.5%, mismatched bases = 4

| | |
|---|---|
| Primer Sequence 5′ to 3′: | CTTCAACAGGACTCACGTGAGCGCGCGCTCATCATCCCATC |
| Complement 5′ to 3′: | GATGGGATGATGAGCGCGCGCTCACGTGAGTCCTGTTGAAG |

TABLE 1
PCR cycle used in site-directed mutagenesis.

| CYCLES | TEMPERATURE | TIME | |
|---|---|---|---|
| 1 | 95° C. | 2 | MINUTES |
| 18 | 95° C. | 20 | SECONDS |
| 18 | 60° C. | 10 | SECONDS |
| 18 | 68° C. | 180 | SECONDS |
| 1 | 68° C. | 5 | MINUTES |

| | |
|---|---|
| ∞ | 4° C. |

After the PCR, 2 μl of the restriction enzyme DpnI are added to each reaction. After mixing gently, each reaction is centrifuged for 1 minute and incubated at 37° C. for 1 hour to allow digestion of the non-mutated DNA to take place. 45 μl of super-competent cells XL10 GOLD and 2 μl of β-ME mix (provided by the kit and stored at −80° C.) are added to the Falcon tubes of polypropylene pre-cooled at 4° C. After mixing gently, the cells are incubated on ice for 10 minutes and mixed gently every 2 minutes. 2 μl of each amplification reaction digested with the enzyme DpnI are added to different aliquots of ultra-competent cells. A plasmid supplied by the kit is used (PUC 18) to check the efficiency of transformation. The cells to which the DNA has been added are incubated for 30 minutes on ice. After this treatment, the cells are ready for transformation by heat shock: the tubes are immersed in a thermostated bath at 42° C. for 30 seconds and then immediately transferred to ice, where they remain for 2 minutes. After adding 0.5 ml of medium LB, each transformation reaction is incubated at 37° C. for 1 hour under stirring (225-250 rpm). The growths are plated on LB/Agar media consisting of LB and Agar (AppliChem) 20 g/l, to which the antibiotic ampicillin (Promega) 1 mM was added, allowing only the growth of cells that have acquired the vector of expression that confers resistance to the antibiotic, and incubated at 37° C. for 18 hours.

### Sequencing of Mutant Plasmids VSAR

Colonies of transformed cells XL10 gold are grown on each of the plates of LB Agar. Two colonies are chosen randomly from the plates of transformations of hproNGF wt VSAR and hproNGF P61SR100E VSAR, from which the plasmid is extracted through mini-preparation and sequenced as described previously. The nucleotide sequences of plasmids Pet11a-hproNGF wt VSAR and Pet11a-hproNGF P61SR100E VSAR obtained by sequencing are compared by nucleotide sequence alignment program BLAST (Basic Local Alignment Search Tool) with the nucleotide sequence the inventors indicated as the one expected after the mutation. The nucleotide sequence using the Translate function of Expasy (Expert Protein Analysis System) is translated into the corresponding amino acid sequence, which is then compared with the one expected, through the alignment function of amino acid sequences Clustal W (Expasy).

### Preparation of Chemically Competent E. Coli Cells for Transformation

E. Coli cells (Rosetta (DE3)) have been sampled from a stock of glycerol stored at −80° C. and inoculated into 1 ml of sterile medium LB. The E. Coli cells of the Rosetta strain (DE3) allow the expression of eukaryotic proteins containing rare codons such as AGG, AGA, AUA, CUA, CCC, GGA, as they produce the transfer RNAs (tRNAs) for these codons on a compatible, chloramphenicol-resistant plasmid. The inoculum is incubated at 37° C. for 18 hours under stirring (200 rpm). 0.5 ml of culture are used to inoculate 25 ml of fresh sterile LB and incubated at 37° C. under stirring until it reaches an optical density measured at 600 nm of 0.4. The culture is cooled on ice for 20 minutes and centrifuged at 5000 rpm for 5 minutes at 4° C. (Beckman, JA-10 rotor). The collected pellet is re-suspended gently in 12.5 ml of a solution of CaCl₂ 0.1 M and incubated for 30 minutes on ice. The inventors then proceeded to a second centrifugation under the same conditions as the previous one. The pellet obtained is re-suspended gently in 2.5 ml of a solution of CaCl₂ 0.1 M. After this treatment, the cells, made competent for transformation, can be transformed within one hour, or they can be stored by adding glycerol up to a final concentration of 10%. After adding the glycerol, the cells are aliquoted in sterile Eppendorf microtubes, preserved in ice for 30 minutes and frozen at −80° C.

### Transformation by Heat Shock of E. Coli (Rosetta (DE3))

A volume of 4 μl (about 40 ng) of the plasmid containing hproNGF wt VSAR and hproNGF P61SR100E VSAR is added to 50 μl aliquots of competent cells previously thawed on ice (the DNA was previously diluted 1:10 from the initial stock). After 2 hours of incubation at 4° C., the cells are subjected to heat shock: the tubes are immersed in a thermostated bath at 42° C. for 45 seconds and then immediately transferred to ice, where they remained for 5 minutes. 400 μl of medium LB are added to the cells transformed in this way, where they are left to grow for 1 hour at 37° C. under stirring. The growths are then centrifuged for 1 minute at 5000 rpm. The pellet, which is re-suspended in a small quantity of supernatant, is plated on a medium LB/Agar, to which the antibiotic ampicillin (Promega) 1 mM has been added. The plates are incubated at 37° C. for 18 hours. The colonies grown on plates are definitely transformed with the plasmid Pet11a that confers resistance to the ampicillin. To obtain the stock in glycerol of Rosetta cells (DE3) containing the two mutated plasmids, individual colonies of each mutant are inoculated in 2 ml of medium LB, to which the inventors previously added the ampicillin (Promega) 1 mM and 35 μg/ml of chloramphenicol (Sigma), and incubated at 37° C. for 18 hours under stirring at 200 rpm. After adding glycerol at 10%, the cells are divided into 1 ml aliquots and stored at −80° C.

Expression and Purification of Mutant Human proNGF Wild-Type VSAR and Human proNGF P61SR100E VSAR

The mutated proteins hproNGF wt VSAR and hproNGF P61SR100E VSAR are produced and purified from the E. Coli cells transformed with the mutated plasmids VSAR using the same procedure described previously for the production and purification of hproNGF wt and hproNGF P61SR100E. The concentration of hproNGF wt VSAR and P61SR100E VSAR is measured with the Nanodrop knowing the molecular weight of the monomer (hproNGF wt VSAR: 2476.1 Da; hproNGF P61SR100E VSAR: 24724 Da) and the molar extinction coefficient: 25355 (same for both proteins).

Protocol Optimisation by Proteolytic Cleavage of Human proNGF with Trypsin for Mutants VSAR
Human proNGF Wild Type VSAR.

### Proteolytic Cleavage Tests in Small Scale

Aliquots of 10 μg of hproNGF wt VSAR and hproNGF wt are incubated with trypsin (Promega) under the conditions specified in Table 2 and then analysed by SDS-PAGE as described herein.

TABLE 2
Conditions used in proteolytic cleavage tests in small scale (hproNGF
wild type VSAR), TRYPSIN:PROTEIN represent weight ratio.

| | INCUBATION | | |
|---|---|---|---|
| TEMPERATURE | TIME | TRYPSIN:PROTEIN | BUFFER |
| 4° C. | overnight | 1:250 | 50 mM sodium |
| | | | phosphate pH 7 |
| 4° C. | overnight | 1:500 | 50 mM sodium |
| | | | phosphate pH 7 |
| 4° C. | overnight | 1:1000 | 50 mM sodium |
| | | | phosphate pH 7 |
| 22° C. | overnight | 1:250 | 50 mM sodium |
| | | | phosphate pH 7 |
| 22° C. | overnight | 1:500 | 50 mM sodium |
| | | | phosphate pH 7 |
| 22° C. | overnight | 1:1000 | 50 mM sodium |
| | | | phosphate pH 7 |
| 16° C. | overnight | 1:1000 | 50 mM sodium |
| | | | phosphate pH 7 |
| 16° C. | overnight | 1:5000 | 50 mM sodium |
| | | | phosphate pH 7 |
| 16° C. | overnight | 1:1000 | 50 mM sodium |
| | | | phosphate pH 7 |

### Proteolytic Cleavage Tests in Large Scale

Aliquots of 3 mg of hproNGF wt VSAR and hproNGF wt are incubated with trypsin (Promega) under the conditions specified in Table 3 and then purified by ion-exchange chromatography as described in section 4.1.9. As for the test carried out at a temperature of 22° C. for 24 hours with trypsin (Promega) at a trypsin:protein ratio of 1:500 at pH 8, hproNGF wt and wt VSAR (3 mg each) were first dialysed against Tris-HCl 50 mM, pH 8.0, then subjected to proteolytic cleavage under the conditions described. The sample is then divided into two aliquots of equal volume. One is purified on a weak anion-exchange resin: Hi Trap DEAE FF 5 ml, GE Healthcare consisting of propylene, which gives positive charge to the resin. Tris-HCl 50 mM, pH 9.7 is used as purification buffer, in which the protein is transferred after a dialysis of 2 hours at 4° C. The column is connected to the instrument for automated purification ÄKTA pure (GE Healthcare) and subjected to balancing through the passage of: 5 column volumes of buffer A (Tris HCl 50 mM, pH 9.7), 5 column volumes of buffer B (Tris-HCl 50 mM, pH 9.7, NaCl 1 M) and 5 column volumes of buffer A. At this point, the sample is loaded. NGF, exposed at a pH greater than its pI, assumes a negative charge that allows its interaction with the positively charged resin. After loading, the resin is washed with two column volumes of buffer A. Elution occurs through the formation of a gradient from 0% to 100% of buffer B in 12 column volumes.

The second aliquot, after being transferred through dialysis in the sodium phosphate buffer 50 mM, pH 7.0, is purified using ion-exchange chromatography as described previously. The fractions corresponding to the peaks eluted by the purification of the proteolytic products of hproNGF wt and wt VSAR are analysed by SDS-PAGE as described previously.

TABLE 3
Conditions used in proteolytic cleavage tests in large scale
(hproNGF wild type VSAR) TRYPSIN:PROTEIN represent
weight ratio

| | INCUBATION | | | |
|---|---|---|---|---|
| TEMPERATURE | TIME | | TRYPSIN:PROTEIN | BUFFER |
| 22° C. | overnight | | 1:1000 | 50 mM |
| | | | | sodium |
| | | | | phosphate |
| | | | | pH 7 |
| 22° C. | 24 | hours | 1:1000 | 50 mM |
| | | | | sodium |
| | | | | phosphate |
| | | | | pH 7 |
| 22° C. | 24 | hours | 1:500 | 50 mM |
| | | | | sodium |
| | | | | phosphate |
| | | | | pH 7 |
| 30° C. | 2 | hours | 1:500 | 50 mM |
| | | | | sodium |
| | | | | phosphate |
| | | | | pH 7 |
| 25° C. | 24 | hours | 1:500 | 50 mM |
| | | | | sodium |
| | | | | phosphate |
| | | | | pH 7 |
| 30° C. | 4 | hours | 1:500 | 50 mM |
| | | | | sodium |
| | | | | phosphate |
| | | | | pH 7 |
| 30° C. | 4 | hours | 1:250 | 50 mM |
| | | | | sodium |
| | | | | phosphate |
| | | | | pH 7 |
| 37° C. | 1 | hour | 1:500 | 50 mM |
| | | | | sodium |
| | | | | phosphate |
| | | | | pH 7 |
| 22° C. | 24 | hours | 1:500 | 50 mm Tris |
| | | | | HCl, pH8 |
| 4° C. | 44 | hours | 1:500 | 50 mM |
| | | | | sodium |
| | | | | phosphate |
| | | | | pH 7 |

### Analysis of Cleavage Intermediates by Mass Spectrometry

In order to identify the band of molecular weight 25 kDa visible on the control acrylamide gel of the proteolytic product of hproNGF wt and mutant wt VSAR, the inventors perform a mass spectrometry analysis. This band is always present in the control gels made after the purification of the proteolytic cleavage reactions under all conditions analysed. 10 μg of proteolytic cleavage product obtained by proteolysis at 25° C. for 24 hours with trypsin (Promega) at a trypsin:protein ratio of 1:500 (the choice was random because the band is present in all conditions) are analysed by SDS-PAGE (as described in section 4.1.10). The bands of interest are cleaved in sterile environment and immersed in H₂O ROMIL in Eppendorf microtubes. They are then subjected to a process of digestion through the following steps:
2 washes (15 minutes each) with H₂O at 37° C. under stirring (350 rpm) using an Eppendorf ThermoMixer
3 washes (20 minutes each) at 37° C. under stirring (350 rpm) with an appropriate volume of washing solution consisting of: 50% Acetonitrile (ACN) and 50% of a solution of 1 M ammonium bicarbonate (FLUKA 09830 MW: 79.06) and CH5NO3 (AMBIC).
Dehydration: the bands are incubated for 5 minutes at 37° C. under stirring (350 rpm) in an Eppendorf ThermoMixer, with Acetonitrile (ACN) at 100%.
Reduction: the bands (dehydrated) are incubated at 56° C. under stirring (350 rpm) for 45 minutes in a solution of 10 mM DTT and 100 mM AMBIC.
Alkylation: the bands are incubated at 37° C. under stirring (350 rpm) for 30 minutes in the dark with a solution of 55 mM iodacetamide (IAA) in 100 mM AMBIC.
2 washes with the washing solution as described above.
Dehydration with ACN 100%.

This treatment has prepared the bands for subsequent digestion with a solution of trypsin 10 ng/μl in 100 mM AMBIC prepared in ice. After adding the trypsin, the bands are incubated for about 16 hours at 37° C. under stirring at 350 rpm. After a quick centrifugation at 5000-6000 rpm, digestion is blocked by adding trifluoroacetic acid (TFA) at 10%. At this point, the fragments of gel are subjected to the following treatments:
Quick centrifugation at 5000-6000 rpm.
Incubation at 37° C. under stirring at 350 rpm for 20 minutes.
Quick centrifugation at 5000-6000 rpm.
Incubation at 37° C. for 20 minutes, under stirring at 350 rpm with 50 μl of a solution consisting of 60% ACN and 0.1% TFA.
Quick centrifugation at 5000-6000 rpm.
Incubation at 37° C. under stirring at 350 rpm for 15 minutes with 50 μl of 100% ACN.
Quick centrifugation at 5000-6000 rpm.
Drying in the concentrator Thermo Scientific™ Savant™ SPD131DDA SpeedVac™
Sonication in the bath for 5 minutes at 100% power, after adding a solution of 3% ACN in 0.1% TFA.
Quick centrifugation at 5000-6000 rpm.
Freezing at −20° C.

The plate for the reading with the MALDI apparatus (Matrix-Assisted Laser Desorption/ionization) is prepared, smearing the wells on which the sample will be spotted with a solution called THIN LAYER (10 mg/ml in CH3CN:EtOH=50:50 and 0.001% TFA). A number of Eppendorf microtubes (0.2 ml) equivalent to the number of samples to be tested are prepared (in our case 3: control, wt and wt VSAR), to which the inventors add 200 μl of solution 2 (0.1% TFA) and other 3 containing 5 μl of solution of HCCA 3.6-5 mg/ml. With the multichannel, preventing air from seeping in, the inventors sample a Zip Tip C18 (Merck Millipore). The resin C18 contained in the tips consists of 18 carbon atom chains and allows both to purify the sample and to further concentrate the small peptides obtained by digestion of the bands. The resin contained in the tip is washed for 5 times with 10 μl of solution 1 (CH3CN/0.1% TFA 1:1) by discharging the tip on precision wipes KIMTECH that are chemically inert and leave no residues. The inventors then proceeded with the balancing: 10 μl of solution 2 (0.1% TFA) are sampled 5 times discharging the tip on precision wipes KIMTECH. After having balanced the tip, the sample to be analysed is passed through the resin of the tip for about 40 times. The resin of the tip is then washed for 9 times with solution 2 (0.1% TFA) by discharging the tip on precision wipes KIMTECH. After the last wash, 2 μl of solution are sampled for elution: 3.5-10 mg/ml in CH3CN:H₂O (50:50) and TFA 0.1% HCCA 3.6-5 mg/ml and the tip is quickly emptied on the well of the plate without touching it. The calibration peptide mix is also spotted on the plate: 2 μl of a solution of 1.5 μl of matrix, HCCA 3.6-mg/ml and 1.5 μl of peptide mix. The peptides identified by MALDI mass spectrometry are compared with the database of human proteins and proteins of E. Coli, using the software MASCOT (www.matrixscience.com) that works using the algorithm MOWSE (MOlecular Weight SEarch), which assigns a probability that the peptides identified correspond to a given protein, attributing a score for each protein identified.

Analysis of Cleavage Intermediates with Western Blot

The products of proteolytic cleavage of hproNGF wt and wt VSAR are loaded on a previously prepared acrylamide gel at 15% under the following conditions:
25° C. for 24 hours with trypsin (Promega) at a trypsin:protein ratio of 1:500 (the same samples used for the mass spectroscopy analysis)
22° C. for 24 hours with trypsin (Promega) at a trypsin:protein ratio of 1:500 in two different quantities: 4 μg and 1 μg. Preparation of the gel and the electrophoretic run take place as described in section 4.1.10. The gel is not coloured though, but the proteins are transferred onto a nitrocellulose membrane (GE Healthcare) using a Trans-Blot® SD Semi-Dry Electrophoretic Transfer Cell (BIORAD). The transfer buffer used consists of: Tris-Base (Sigma) 25 mM, glycine (AppliChem) 192 mM, pH 8.3, to which methanol (VWR Prolabo BDH chemicals) at 20% is added right before use. The transfer takes place in about 50 minutes at 70 mA at a constant amperage. The nitrocellulose membrane is stained with Ponceau red dye (Applichem) to make sure the transfer has occurred. To prevent the antibody from binding to non-specific binding sites, the sites are blocked by incubating the membrane for 1 hour, stirring it at room temperature with a solution of 4% milk (Applichem) in PBS (Phosphate Buffered Saline) to which the TWEEN detergent was added to obtain a final concentration of 0.05% (Composition PBS 10×: NaCl 1.37 M, KCl 27 mM, Na₂HPO₄H₂O 81 mM, KH₂PO₄ 19 mM, pH 7.4). After this blocking step, the membrane is incubated at 4° C. for about 18 hours with the primary antibody (polyclonal antibody produced in rabbit anti-proNGF, CHEMICON) diluted in 4% milk PBS/TWEEN 4% milk to obtain a final concentration of 1:200. The membrane is then subjected to 4 washes of 10 minutes each with PBS/TWEEN 0.05%. At this point, the inventors proceeded with incubation for 1 hour under stirring at room temperature with HRP-conjugated secondary antibody (anti-rabbit, Jackson Lab) diluted in 4% milk PBS/TWEEN 0.05% to obtain a final concentration of 1:7000. After a series of 4 washes lasting 10 minutes each with PBS/TWEEN 0.05%, the membrane is developed using a commercial chemiluminescence kit Amersham ECL Select detection reagent kit (GE Healthcare) comprising two reactants: a solution of luminol and a peroxidase solution. The peroxidase, which is conjugated with the secondary antibody, oxidises the luminol that produces the chemiluminescent signal. The signal is detected using KODAK image station 2000R, an imaging tool that enables an analysis in chemiluminescence, fluorescence, densitometry, chromogenic marking and radioactivity of membranes, gels, plates and tissues.

The primary and secondary antibody are then removed from the membrane by using a procedure called stripping that includes: incubation for 15 minutes at room temperature, under stirring with stripping buffer (SDS (Applichem) 10%, Tris HCl 0.5 M, pH 6.8, ddH₂O) previously heated, to which fresh DTT (Applichem) 0.1 M is added, followed by two washes with stripping buffer without the addition of DTT. After a thorough washing with H₂O to eliminate the DTT, the analysis is repeated in the same way using a polyclonal primary antibody produced in rabbit anti-NGF (H-20, Santa Cruz) diluted in 4% milk PBS/TWEEN 0.05% to obtain a final concentration of 1:200.

Human proNGF P61SR100E VSAR

### Proteolytic Cleavage Tests in Small Scale

Aliquots of 120 μg of hproNGF P61SR100E VSAR and hproNGF P61SR100E are incubated with trypsin (Promega) under the conditions specified in Table 4 and then analysed by SDS-PAGE as described above.

TABLE 4
Conditions used in proteolytic cleavage tests in small scale (hproNGF
P61SR100E VSAR), TRYPSIN:PROTEIN represent weight ratio

| | INCUBATION | | |
|---|---|---|---|
| TEMPERATURE | TIME | TRYPSIN:PROTEIN | BUFFER |
| 4° C. | 15′, 30′, 45′, | 1:250 | 50 mM sodium |
| | 1 hour | | phosphate pH 7 |
| 4° C. | 15′, 30′, 45′, | 1:1000 | 50 mM sodium |
| | 1 hour | | phosphate pH 7 |
| 4° C. | 90′, 2 hours, | 1:250 | 50 mM sodium |
| | 4 hours, | | phosphate pH 7 |
| | overnight | | |
| 4° C. | 90′, 2 hours, | 1:1000 | 50 mM sodium |
| | 4 hours, | | phosphate pH 7 |
| | overnight | | |
| 4° C. | 24 hours | 1:1000 | 50 mM sodium |
| | | | phosphate pH 7 |
| 4° C. | overnight | 1:1000 | 50 mM sodium |
| | | | phosphate pH 7 |
| 4° C. | overnight | 1:5000 | 50 mM sodium |
| | | | phosphate pH 7 |
| 4° C. | 24 hours | 1:1000 | 50 mM sodium |
| | | | phosphate pH 7 |
| 4° C. | 24 hours | 1:5000 | 50 mM sodium |
| | | | phosphate pH 7 |
| 30° C. | 1 hour | 1:1000 | 50 mM sodium |
| | | | phosphate pH 7 |
| 30° C. | 1 hour | 1:5000 | 50 mM sodium |
| | | | phosphate pH 7 |
| 20° C. | 4 hours | 1:1000 | 50 mM sodium |
| | | | phosphate pH 7 |
| 16° C. | overnight | 1:1000 | 50 mM sodium |
| | | | phosphate pH 7 |
| 16° C. | overnight | 1:5000 | 50 mM sodium |
| | | | phosphate pH 7 |
| 16° C. | overnight | 1:5000 | 50 mM sodium |
| | | | phosphate pH 7 |
| 16° C. | 24 hours | 1:5000 | 50 mM sodium |
| | | | phosphate pH 7 |

### Proteolytic Cleavage Tests in Large Scale

Aliquots of 3 mg and 13 mg of hproNGF P61SR100E VSAR are incubated with trypsin (Promega) under the conditions specified in Table 5 and then purified by ion-exchange chromatography as described above.

TABLE 5
Conditions used in proteolytic cleavage tests in large scale (hproNGF
P61SR100E VSAR), TRYPSIN:PROTEIN represent weight ratio

| TEMPERATURE | INCUBATION TIME | TRYPSIN:PROTEIN |
|---|---|---|
| 4° C. | Overnight | 1:1000 |
| 16° C. | 24 hours | 1:5000 |

The fractions corresponding to peaks eluted from the purifications are subjected to analysis on SDS-PAGE as described above.

### NGF-Dependent Cell Proliferation Assay on TF1

The biological activity of hNGF produced by the cleavage of the mutant VSAR is tested using a cell proliferation assay on TF1 cells. The TF1 cells are a human hematopoietic cellular line that expresses the TrkA receptor of NGF but not p75NTR. Administration of the exogenous NGF induces autophosphorylation of the TrkA receptor, triggering a cell proliferation signal. For their ability to respond to exogenous NGF by proliferating, they are a good quantitative assay to assess the functionality of hNGF produced in the laboratory. The TF1 cells are left to grow for a week in a culture medium RPMI-1640 containing 10% FBS (fetal bovine serum, Gibco) and 2 ng/ml of GM-CSF (Granulocyte-macrophage colony-stimulating factor). The cells are then washed and re-suspended in RPMI-1640 (Gibco) containing 10% FBS (Gibco) so as to obtain a final concentration of 300,000 cells/ml. 15000 cells per well are plated on a plate with 96 wells in a volume of 50 μl. An hour later, the cells are exposed to the hNGF to be tested, previously diluted appropriately to obtain the following dilutions: 200, 100, 75, 50, 25, 10, 7.5, 5, 1, 0.5, 0.1 ng of hNGF. In some wells, NGF is not added (which will represent the zero curve). Each treatment, which has a different concentration of NGF administered, is repeated in duplicate. After 40 hours of incubation at 37° C. at 5% carbon dioxide (CO₂), the culture medium (RPMI-1640 containing 10% FBS (Gibco)) is changed and 20 μl of reagent The CellTiter 96® AQᵤₑₒᵤₛ One Solution (Promega), stored at −20° C., containing a tetrazolium compound [3-(4,5-dimethylthiazol-2-yl)-5-(3-carboxymethoxyphenyl)-2-(4-sulfophenyl)-2H-tetrazolium are added to each well. This reagent allows us to determine the number of viable cells in proliferation using a colorimetric assay. After adding the reagent, the plates are incubated at 37° C. for a time between 1 and 3 hours in a humidified environment at 5% CO₂. The absorbance is read at a wavelength of 490 nm after 1, 2 and 3 hours of incubation using an ELISA reader (Bio-rad).

### Site Specific Mutagenesis of Single Mutants

The point mutations Val101Arg e Ala103Lys were inserted in the plasmids pET11a, containing the ORF (open reading frame) of hproNGF P61S, hproNGF R100E. The mutations were carried out by using the commercial kit QuikChange II XL Site-Directed Mutagenesis (Agilent Technologies) and following the manufacturer's instruction. The used primers were:

(SEQ ID NO: 10)
CTTCAACAGGACTCACGTGAGCGCGCGCTCATCATCCCATC
(SEQ ID NO: 11)
GATGGGATGATGAGCGCGCGCTCACGTGAGTCCTGTTGAAG

For all the plasmids.

The mutagenesis reactions were used to transform the supercompentent cells XL10 GOLD, supplied by the kit. The plasmids were extracted by the positive colonies and sequenced in order to assess the good incorporation of the point mutations. The E. coli strain Rosetta (DE3)pLysS was transformed with the obtained plasmids: pET11a hproNGF P61S VSAR, pET11a hproNGF R100E VSAR.

### Expression and Purification of VSAR Single Mutants

hproNGF P61S VSAR, hproNGF R100E VSAR were expressed in E. coli Rosetta (DE3)pLysS and purified as previously described for the non VSAR mutants (Malerba et al 2015). Briefly, proNGF was resolubilized from E. coli inclusion bodies and refolded. The purification was carried out by two consecutive chromatographies: an anionic exchange and a hydrophobic interaction.

hproNGF P61SR100E VSAR Mass Spectrometry

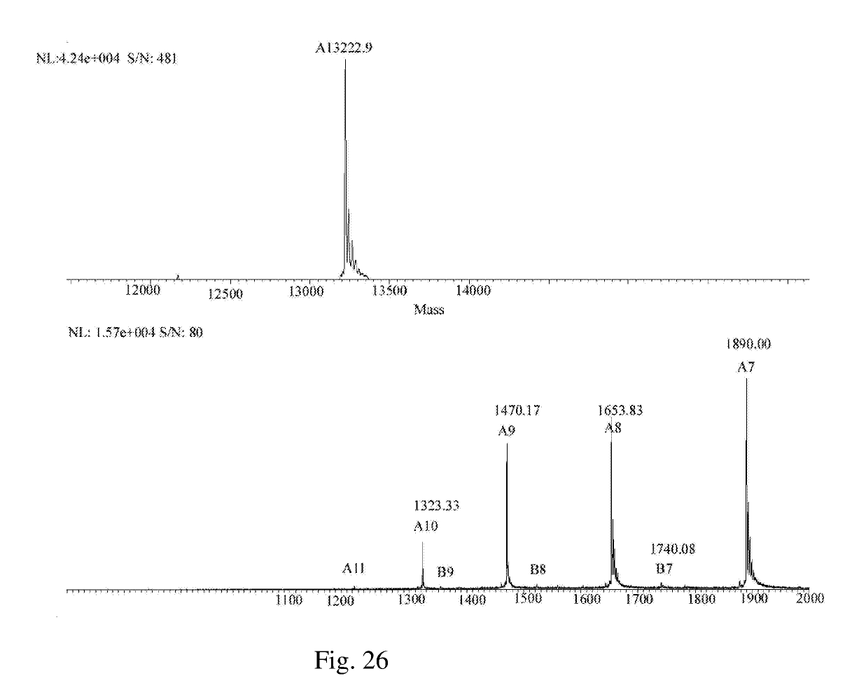

hNGF P61SR100E, cleaved from hproNGF P61SR100E VSAR, following this condition: 16° C. 24 h trypsin:protein=1:5000, was analyzed by MALDI TOF Mass Spectrometry (MS), in order to assess the molecular weight and the molecular species present (FIG. 26).

hproNGF P61S VSAR and hproNGF R100E VSAR Proteolytic Cleavage

In order to compare the result obtained with hproNGF WT VSAR and hproNGF P61SR100E, experiments with the single mutants (hproNGF P61S VSAR and hproNGF R100E VSAR) were carried out.

120 μg of hproNGF P61SR100E, hproNGF P61S VSAR and hproNGF R100E VSAR (concentration 0.6 mg/ml) were incubated with trypsin (promega) at the conditions indicated in table 4a and then analyzed directly by Mass Spectrometry.

TABLE 4a
The two cleavage protocols tested on hproNGF P61SR100E,
hproNGF P61S VSAR and hproNGF R100E VSAR (120 μg)

| TEMPERATURE | INCUBATION | TRYPSIN:PROTEIN RATIO |
|---|---|---|
| 4° C. | 24 h | 1:10000 |
| 16° C. | 24 h | 1:5000 |

The best condition (16° C. 24 h weight ratio trypsin:protein=1:5000) was chosen to carry out cleavage experiments at a larger scale. 3 mg (concentration 0.6 mg/ml) of hproNGF P61SR100E, hproNGF P61S VSAR and hproNGF R100E VSAR were incubated with trypsin (Promega), then purified by ionic exchange chromatography (Hi TRAP SP Sepharose XL GE Healthcare) The positive fractions were collected, concentrated and analyzed by Mass Spectrometry.

In order to explore other cleavage conditions, listed in table 5a, 3 mg of hproNGF P61SR100E, hproNGF P61S VSAR and hproNGF R100E VSAR were incubated with trypsin (Promega), then purified by ionic exchange chromatography (Hi TRAP SP Sepharose XL GE Healthcare) The positive fractions were collected, concentrated and analyzed by Mass Spectrometry.

TABLE 5a
Other cleavage protocols tested on hproNGF P61SR100E,
hproNGF P61S VSAR and hproNGF R100E VSAR (3 mg).

| | | TRYPSIN:PROTEIN |
|---|---|---|
| TEMPERATURE | INCUBATION | WEIGHT RATIO |
| 16° C. | 17 h | 1:5000 |
| 16° C. | 4 h | 1:5000 |
| Room | 4 h | 1:5000 |
| Temperature* | | |

*This condition was tested only on hproNGF P61SR100E.

Side by Side hproNGF P61SR100E VSAR Versus hproNGF WT VSAR Comparison

3 mg of hproNGF P61SR100E VSAR and hproNGF WT VSAR (concentration 0.6 mg/ml) were digested in parallel, using the same reagents and identical conditions, at the same time, by following these two protocols:

1) Room Temperature, 18 h, trypsin:protein weight ratio=1:10000;
2) 16° C., 24 h, trypsin:protein weight ratio=1:5000;
and then purified by ionic exchange chromatography (Hi TRAP SP Sepharose XL GE Healthcare). The positive fractions were collected and concentrated. 10 μg of each purified sample were run on a SDS PAGE (15% Polyacrylamide) and colored by EzWay™ Protein-Quick Blue Staining Solution (KOMABIOTECH).

### Examples

### Expression and Purification of Human NGF Wild-Type and Human Mutant NGF P61SR100E

Purification of Human proNGF Wild Type.

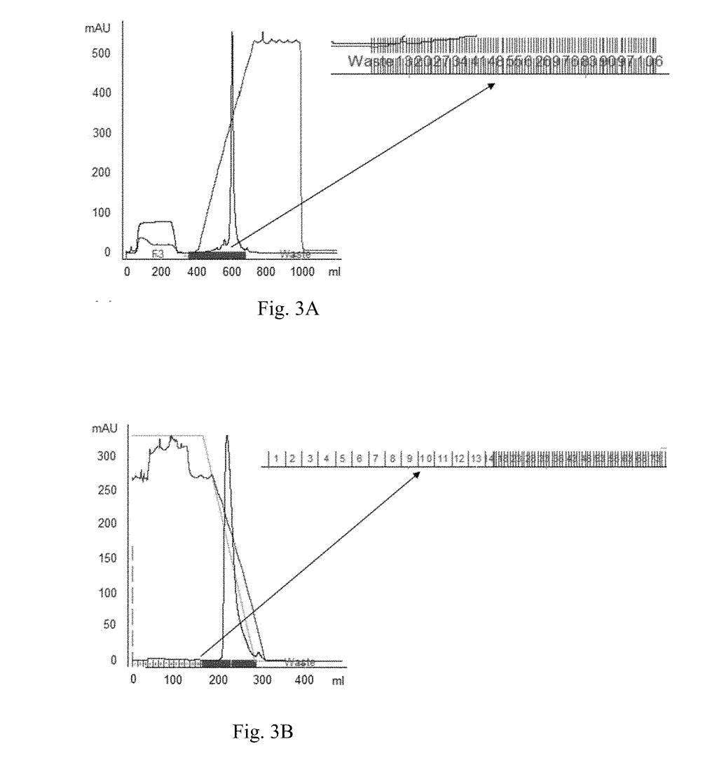

As described in the materials and methods, the purification of hproNGF wt takes place after isolation and solubilisation of the inclusion bodies and subsequent refolding. The chromatographic part includes two subsequent chromatographies: ion-exchange and hydrophobic interaction. In both chromatographies (FIG. 3), the hproNGF wt is eluted as a peak in the second half of the gradient. The peak obtained by ion-exchange chromatography is eluted at an ionic strength of 53 mS/cm, while the hydrophobic-interaction chromatography at the point of maximum height corresponds to an ionic strength of 101 mS/cm. The peak fractions are recovered and merged. The concentration of purified hproNGF wt is: 0.66 ml in a volume of 62 ml. The yield of hproNGF wt obtained from a bacterial growth of 1.7 litres is: 24 mg/l.

Purification of Human proNGF P61SR100E.

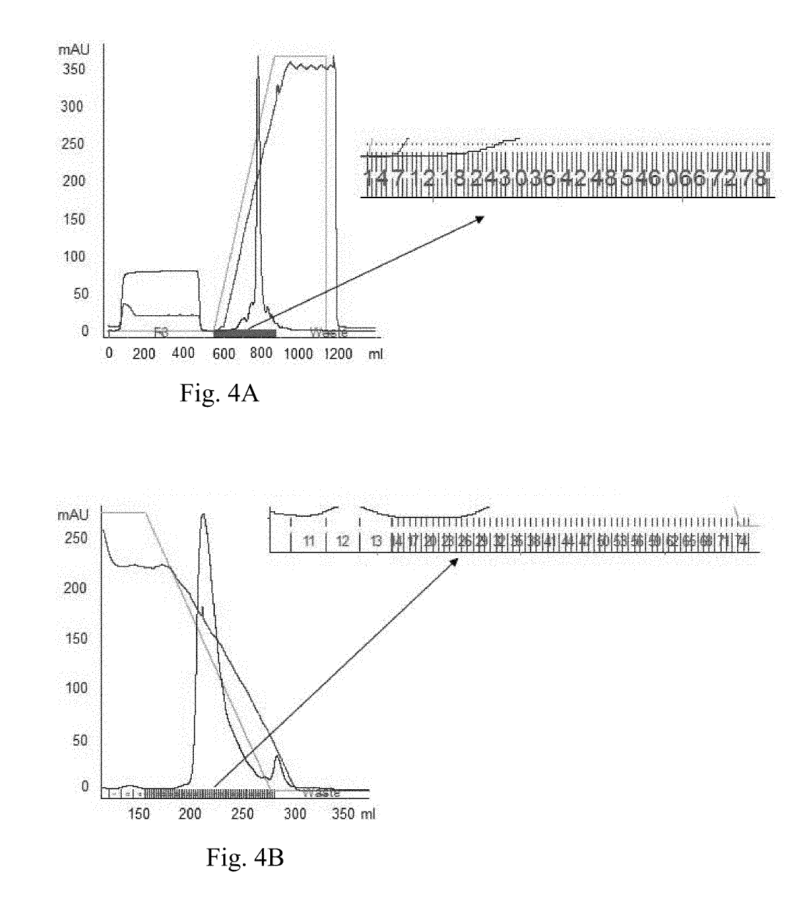

The hproNGF P61SR100E is eluted in the second half of the gradient in both chromatographies (FIG. 4). The peaks obtained by ion-exchange chromatography and hydrophobic-interaction chromatography are eluted with an ionic strength of 50 mS/cm and 103 mS/cm, respectively. The concentration of hproNGF P61SR100E is: 0.6 ml in a total volume of 60 ml of purified protein. The yield of hproNGF P61SR100E obtained from a bacterial growth of 3.4 litres is therefore: 10.6 mg/l.

Purification of Human NGF Wild Type.

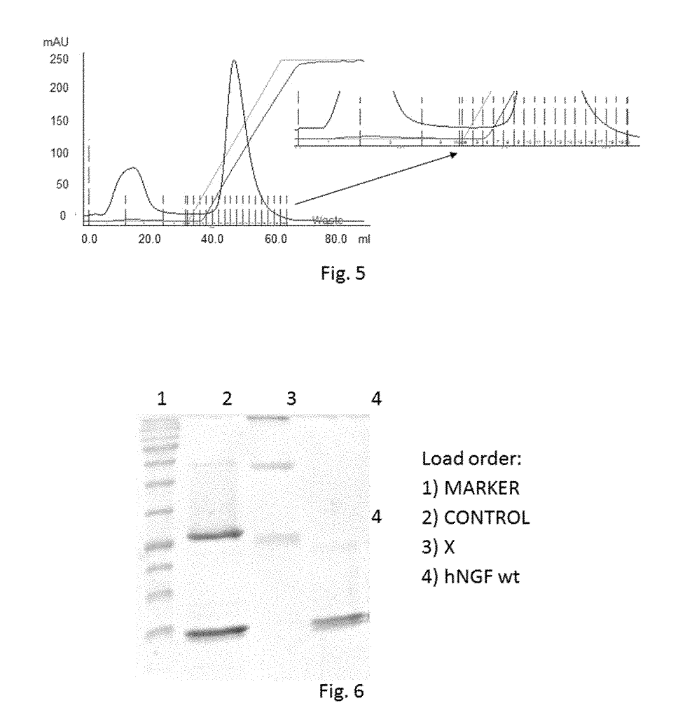

The chromatogram resulting from purification of the proteolytic product of hproNGF wt (obtained by incubation with trypsin at a trypsin:hproNGF wt ratio of 1:150 for 18 hours at 16° C.) is shown in FIG. 5. The peak is eluted in the second half of the gradient at an ionic strength of 29 mS/cm. The subsequent analysis of the proteolytic product by SDS-PAGE (FIG. 6) shows the presence of a band of intermediate molecular weight between NGF and its precursor, indicating that the proteolysis of hproNGF wt was not comprehensive. The NGF molecule has two polypeptide chains: each with a molecular weight of 13,250 kDa merged to form a dimer of 26,500 kDa. The denaturing and reducing conditions of the electrophoretic run cause the detachment of the two monomers, so that the one that runs on the gel is the monomer of NGF, represented by a band of about 13 kDa. The same holds true for hproNGF, which is a dimer of about 50 kDa. What the inventors see on the gel in denaturing conditions is the monomer, which is represented by a band of about 30 kDa. Despite its molecular weight is about 25 kDa, the proNGF is slowed down in the electrophoretic run because of its strong basicity. The concentration of hNGF wt is 0.63 mg/ml in a total volume of 1.5 ml of protein obtained from proteolytic cleavage. The yield obtained by proteolysis of 3 mg of hproNGF wt, given by the ratio between the quantity of NGF obtained (0.9 mg) and the quantity of cleaved proNGF is 0.3.

Purification of Human NGF P61SR100E.

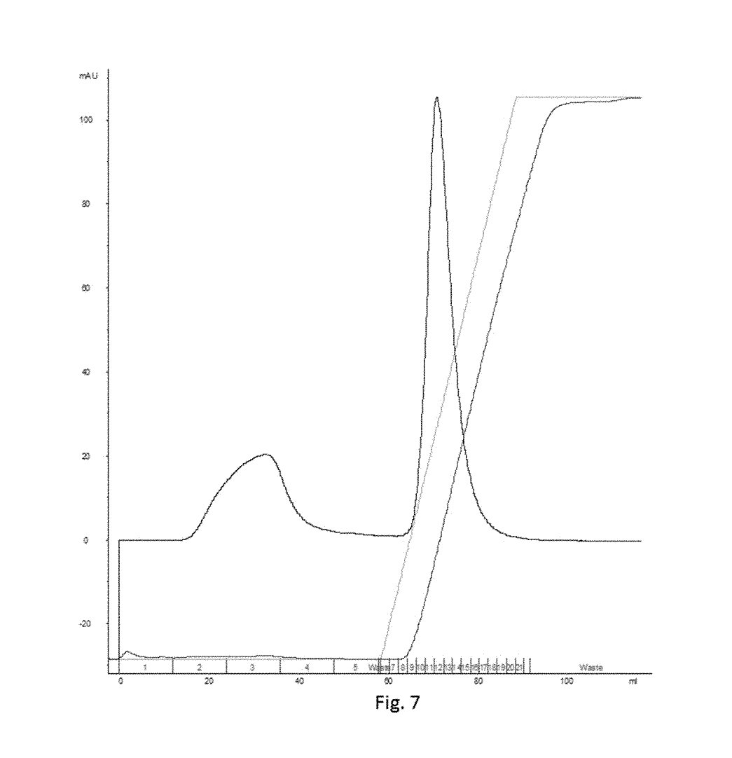

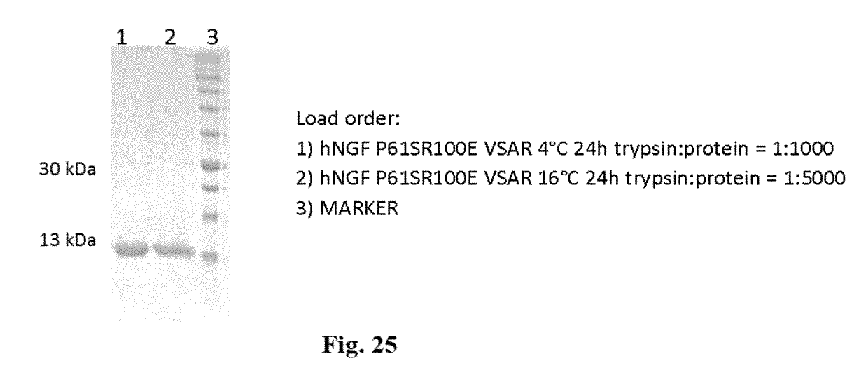

The chromatogram resulting from purification of the proteolytic product of hproNGF P61SR100E (obtained by incubation with trypsin at a trypsin:hproNGF P61SR100E ratio of 1:250 for 1 hours at 30° C.) is shown in FIG. 7. The peak is eluted in the second half of the gradient at an ionic strength of 20 mS/cm. The result of the analysis of proteolytic cleavage product by SDS-PAGE is shown in FIG. 25. The gel was loaded with an excess of protein (10 μg) in order to detect impurities in low concentration: the presence of only one band at a height of about 13 kDa, corresponding to hNGF P61SR100E, shows that the proteolytic cleavage occurred in an efficient manner. The concentration of hNGF P61SR100E is 0.68 ml in a volume of 28 ml (19.04 mg total). The yield obtained by proteolysis of 43 mg of hproNGF P61SR100E, given by the ratio between the quantity of NGF obtained (19.04 mg) and the quantity of cleaved proNGF is 0.4.

### Site-Directed Mutagenesis: Production of Mutants VSAR

### Transformation of E. Coli Cells (Rosetta (DE3)).

In order to make sure that mutagenesis occurred as desired, the DNA sequences obtained have been sent to the Genechron DNA sequencing service (www.genechron.com) to be sequenced. Sequence analysis confirmed the correct incorporation of mutation for both plasmids. The inventors therefore proceeded with the transformation of E. Coli cells (Rosetta (DE3)) with the plasmid Pet11a-hproNGF wt VSAR and Pet11a-hproNGF P61SR100E VSAR. The transformations have resulted in the formation of colonies on medium LB/Agar with ampicillin.

Expression and Purification of Mutant Human proNGF Wild-Type VSAR and Human proNGF P61SR100E VSAR.

hproNGF wt VSAR and hproNGF P61SR100E VSAR proteins are expressed and purified using the same protocol of hproNGF wt and P61SR100E proteins to make sure that there are no changes in bacterial growth, in protein expression, in the efficiency of purification and the final yield of the corresponding proNGF compared to the non-mutated proteins.

Purification of Human proNGF Wild Type VSAR.

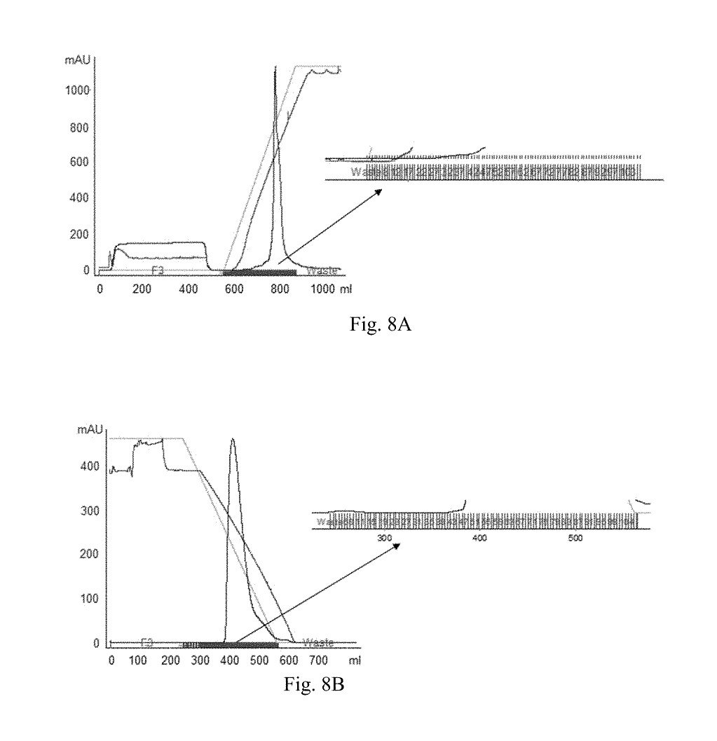

The hproNGF wt VSAR is eluted in the second half of the gradient in both chromatographies (FIG. 8). The peaks obtained by ion-exchange chromatography and hydrophobic-interaction chromatography are eluted with an ionic strength of 49 mS/cm and 65 mS/cm. The inventors obtained 120 ml of hproNGF wt VSAR at a concentration of 0.97 mg/ml and 118 ml of hproNGF wt VSAR at a concentration of 0.96 mg/ml from two different bacterial growths of 3.4 litres each. The yield of the production of hproNGF wt VSAR obtained from the average of the yields obtained from the two growths is 33.8 mg/l.

Purification of Human proNGF P61SR100E VSAR.

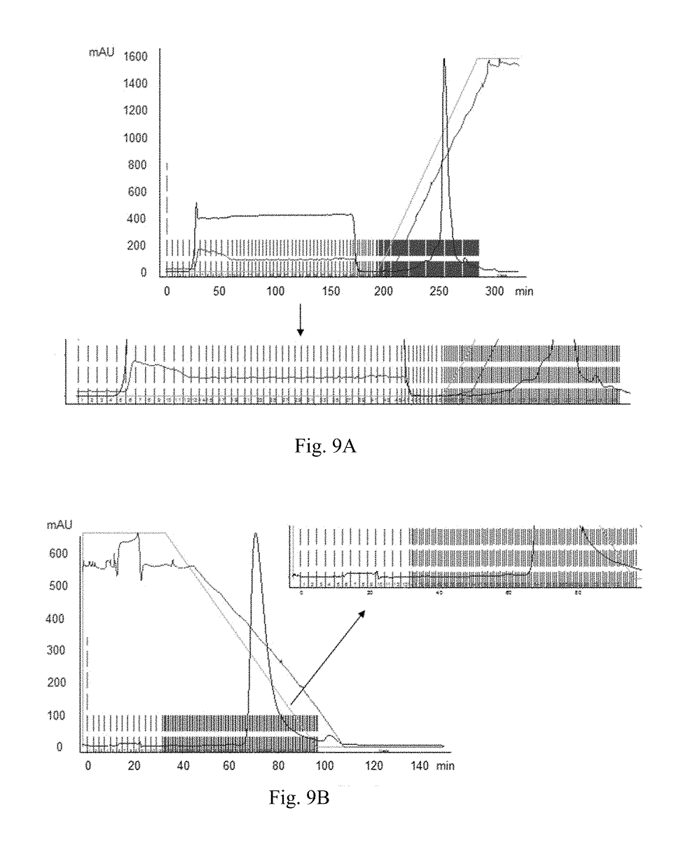

The peaks obtained by ion-exchange chromatography and hydrophobic-interaction chromatography of hproNGF P61SR100E VSAR are eluted with an ionic strength of 42 mS/cm and 76 mS/cm (FIG. 9). The inventors obtained 114 ml of hproNGF P61SR100E VSAR at a concentration of 0.24 mg/ml and 117 ml of hproNGF P61SR100E VSAR at a concentration of 0.20 mg/ml from two different bacterial growths of 3.4 litres each. The yield of the production of hproNGF wt VSAR obtained from the average of the yields obtained from the two growths is 7.5 mg/l.

### Comparison of the Yields of Mutant Human PRONGF Wild-Type and P61SR100E VSAR and Yields of Human PRONGF Wild-Type and P61SR100E.

The final yield of the production of hproNGF wt and P61SR100E is, on average, equal to 35 mg/l for hproNGF wt and 8.3 mg/l for hproNGF P61SR100E. The yield obtained from the production of mutants VSAR which is, on average, equal to 33.8 mg/l for hproNGF wt VSAR and 7.5 mg/l for hproNGF P61SR100E VSAR, can be considered almost unchanged (Table 1a). Arg101Val and Lys103Ala mutations in the consensus sequence for the proteolytic cleavage of proNGF in NGF do not affect the expression, purification and yield of recombinant protein production.

TABLE 1a
Comparison of the yields of mutants hproNGF wt and P61SR100E
VSAR and of the yields of non-mutated hproNGF wt P61SR100E.

| | hproNGF | hproNGFwt | hproNGF | hproNGF |
|---|---|---|---|---|
| | wt | VSAR | P61SR100E | P61SR100E VSAR |
| YIELD mg/l | 35.0 | 33.8 | 8.3 | 7.5 |

Protocol Optimisation by Proteolytic Cleavage of Human PRONGF with Trypsin for Mutants VSAR
Human proNGF Wild Type VSAR.

Extensive tests were carried out by proteolytic cleavage by changing the parameters such as the trypsin:protein ratio, temperature, incubation times and pH, in order to find the best conditions for the mutant VSAR proteolysis. Each condition was compared with non-VSAR protein used as a control.

### Proteolytic Cleavage Tests in Small Scale.

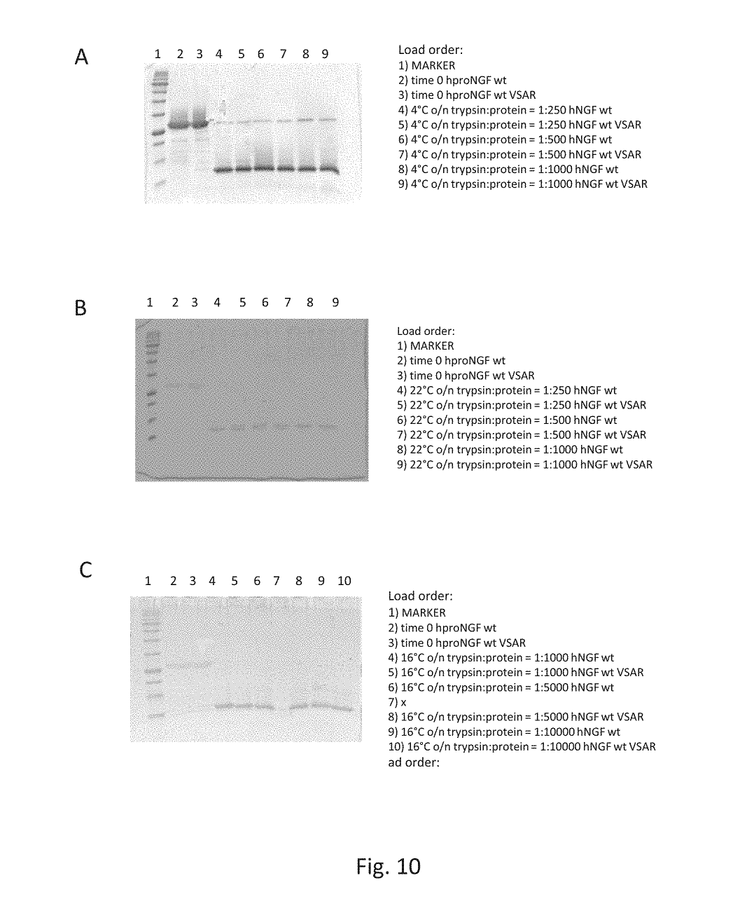

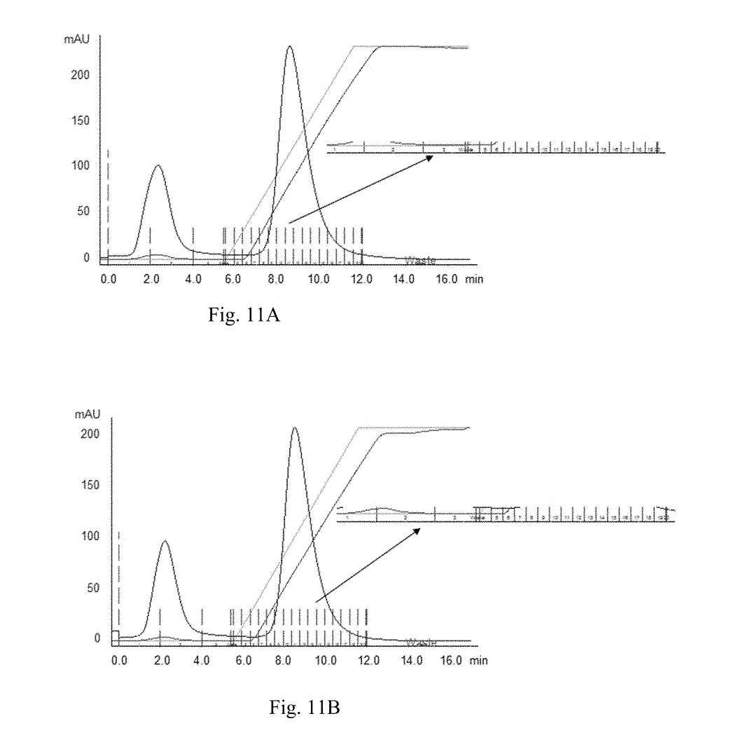

The results of the analysis by SDS-PAGE of the product by proteolytic cleavage mediated by trypsin of aliquots of 10 μg of hproNGF wt ad hproNGF wt VSAR are shown in FIGS. 10 a, b, c. After incubation at 4° C., proteolysis did not occur efficiently in none of the conditions tested. In fact, in FIG. 10 a, in addition to the band of approximately 13 kDa corresponding to NGF, the inventors detect the presence of the band of 30 kDa corresponding to proNGF. The result of the electrophoretic run of the cleavage product obtained following incubations with diminishing concentrations of trypsin (trypsin:protein ratio of 1:250, 1:500, 1:1000) at a temperature of 22° C. is shown in FIG. 10 b. Proteolysis occurred efficiently for both the mutant VSAR and for the non-mutated control, which seem to behave the same way. In this gel, the inventors can observe the presence of a band below the one of 13 kDa corresponding to NGF. This second band might be the so-called desocta NGF reported in the literature as a form of biologically active NGF that is naturally present in the salivary glands of the rat (Holland et al., 1994). After incubations at very low concentrations of trypsin (trypsin:protein ratio of 1:1000, 1:5000, 1:10000), at a temperature of 16° C., in addition to the presence of the band corresponding to NGF, the inventors also detect the one corresponding to proNGF and, especially in incubations with trypsin at a trypsin:protein ratio of 1:5000 and 1:10000 (FIG. 10, lanes 6, 8, 9, 10), the presence of an intermediate form of proteolytic cleavage.

### Proteolytic Cleavage Tests in Large Scale.

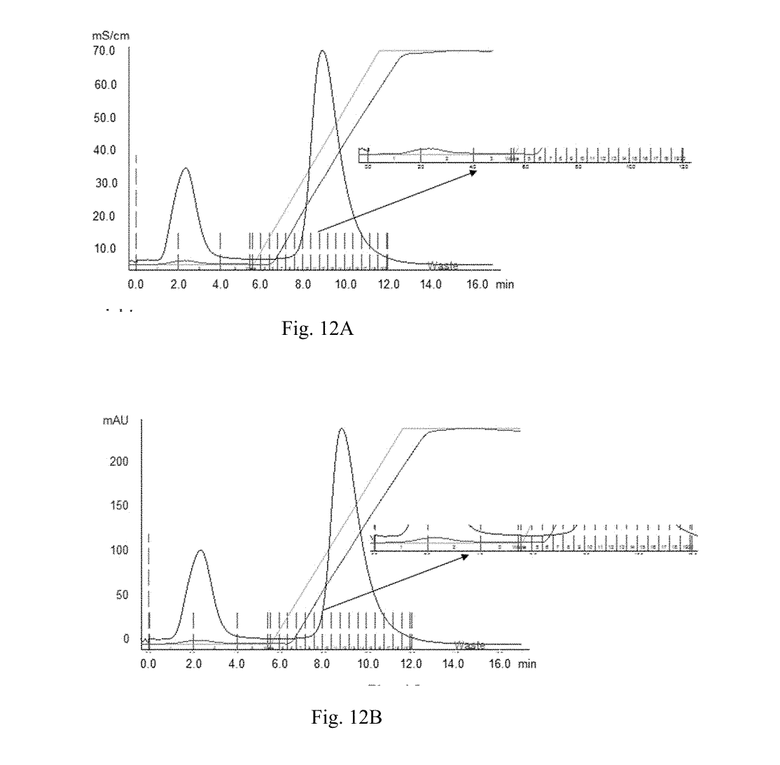

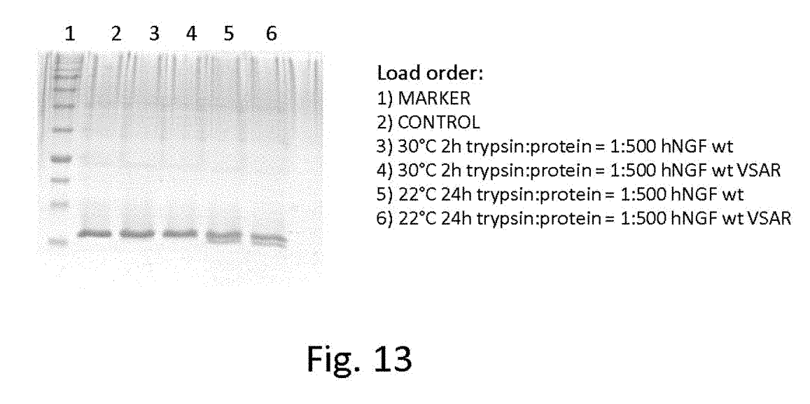

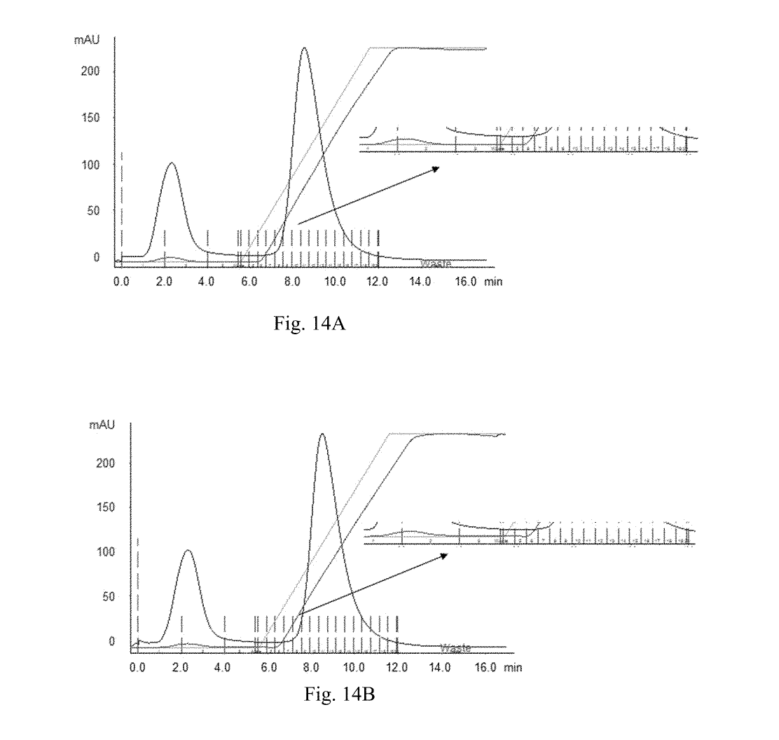

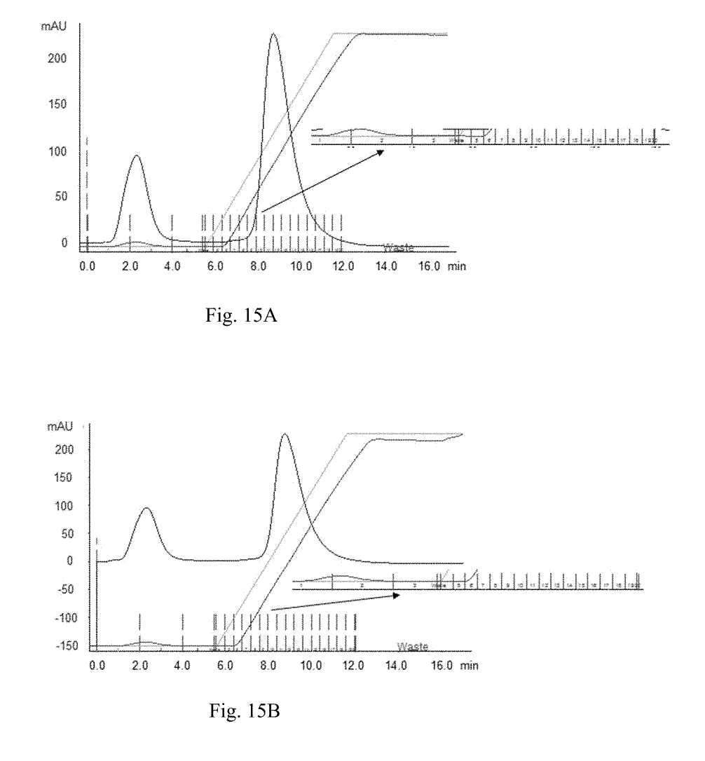

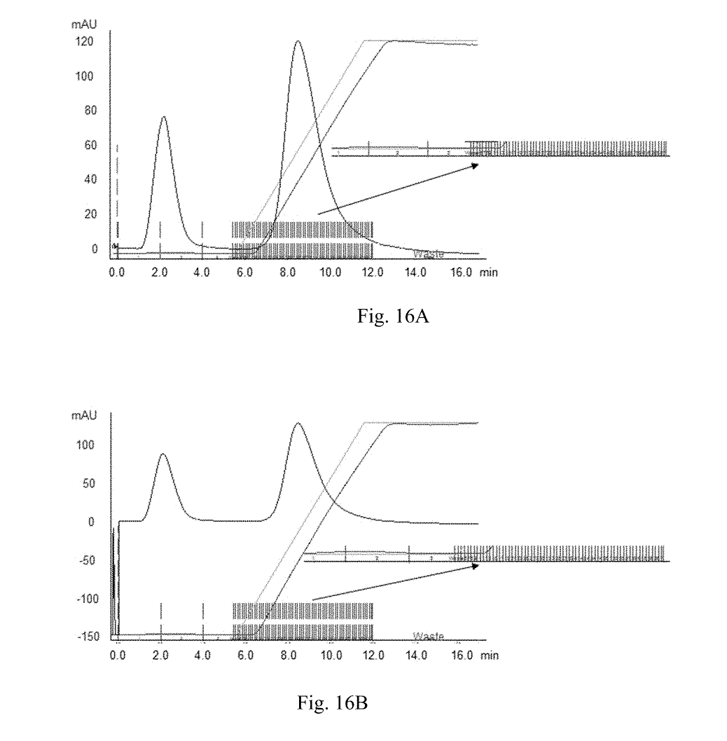

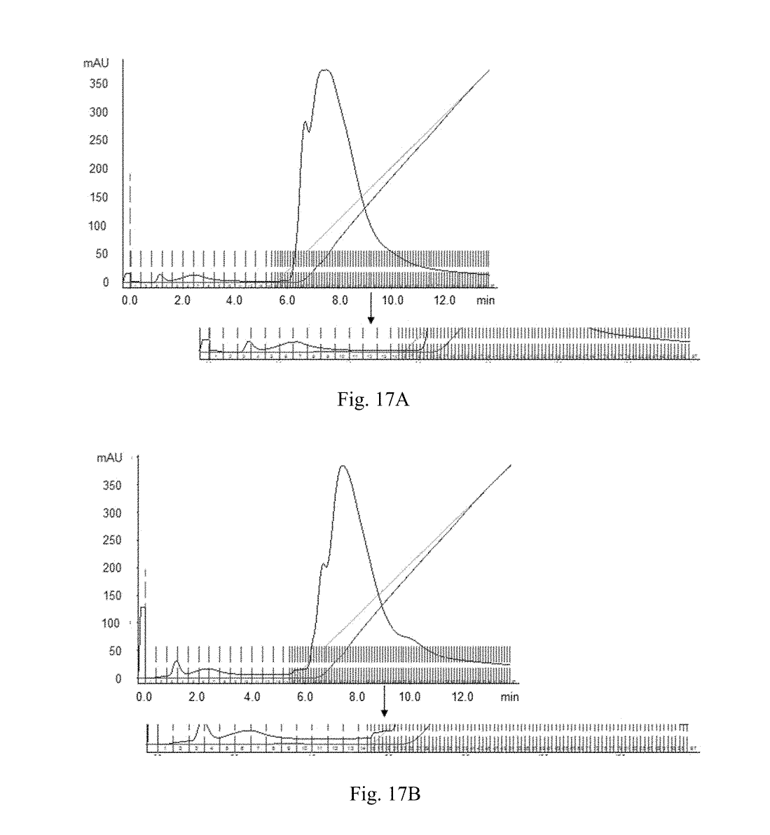

Because by increasing the scale, the reaction conditions used will not necessarily all be efficient in the same way, the inventors decided to test the different conditions of proteolysis (Table 3 of methods and materials) on larger quantities of protein: 3 mg of hproNGF wt and wt VSAR subsequently purified by ion-exchange chromatography. The analysis by SDS-PAGE of the proteolytic cleavage product obtained following incubation with trypsin under all conditions listed in the table showed the presence of a band at the height of 25 kDa, presumably a partially proteolysed form of proNGF, as well as the one of 13 kDa corresponding to NGF. FIGS. 11-17 show some of these results. The band at the height of 25 kDa, varying in intensity, is present in the proteolytic product of hproNGF wt and hproNGF wt VSAR obtained from all the conditions of proteolysis tested. The band is also present in the control: proteolytic product of hproNGF wt obtained following incubation with trypsin at 16° C. for 24 hours at a trypsin:protein ratio of 1:150. Among all those tested, incubation at 30° C. for 2 hours with trypsin at a trypsin:protein ratio of 1:500 seems to be the best, because, as you can see in FIG. 13 (lanes 3 and 4), the intensity of the residual band at 25 kDa is much lower than the one found in the other conditions and the inventors do not detect the presence of desocta NGF.

Incubation of 3 mg of hproNGF wt VSAR at 30° C. for 2 hours with trypsin at a trypsin:protein ratio of 1:500 produced 2.5 ml of hNGF wt at a concentration of 0.44 mg/ml (total 1.1 mg of protein). The yield obtained by proteolysis of 3 mg of proNGF in NGF given by the ratio between the quantity of NGF obtained and the quantity of proteolysed proNGF is 0.36. The incubation under the same conditions of 3 mg of hproNGF wt produced 2.1 ml of hNGF wt at a concentration of 0.45 mg/ml (total 0.95 mg of protein). The yield obtained by proteolysis of 3 mg of proNGF in NGF given by the ratio between the quantity of NGF obtained and the quantity of proteolysed proNGF is 0.31.

### Analysis of Cleavage Intermediates by Mass Spectrometry.

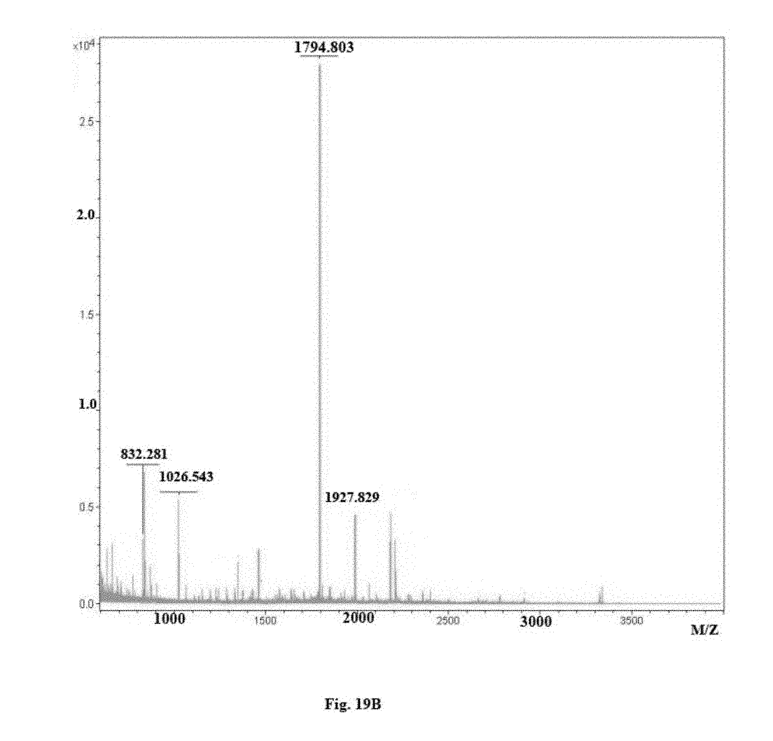

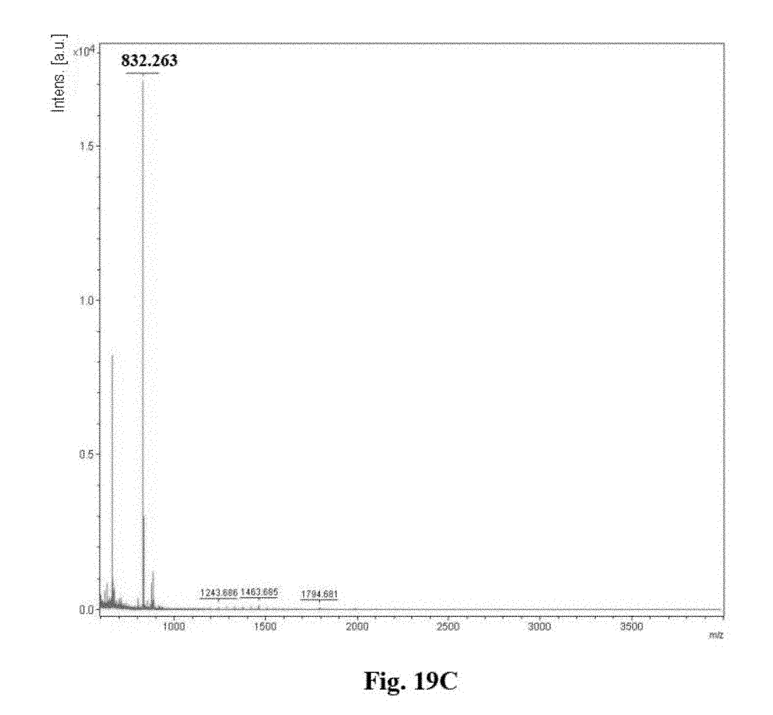

To make sure that the band of 25 kDa is a form resulting from incomplete proteolysis of proNGF in NGF and not a contaminant protein not eliminated during purification, the band was analysed by mass spectrometry. The peptides identified by MALDI in the bands of 25 kDa (FIGS. 19 a, b, c) obtained from the analysis of the proteolytic product of hproNGF wt and wt VSAR following incubation at 25° C. for 24 hours at a trypsin:protein ratio of 1:500 (the choice was random because the band is present in all conditions) and of the control were compared using the software MASCOT (www.matrixscience.com), both with the database of human proteins (the protein is human) and with the database of proteins of E. Coli, which is the bacterium in which the protein was produced. The results obtained are shown in Table 2a. Scores higher than 40 can be considered to be significant. Mass spectrometry analysis identifies peptides belonging to human NGF only in the band at the height of 25 kDa derived from proNGF wt and not, instead, in the one derived from proNGF wt VSAR.

TABLE 2a
Proteins identified from comparison of peptides identified by mass
spectrometry in the band of 25 kDa (present in the proteolytic product
of hproNGF wt and wt VSAR) with the database of
human and E. Coli proteins using the algorithm MASCOT.

| | | | SWISSPROT | Mascot |
|---|---|---|---|---|
| Spot | | | Accession | PMF |
| Number | Protein name | DATABASE | Number | score |
| CTRL | Beta-lactamase SHV-34 | E. COLI | CHEZ_ECOBD | 65 |
| VSAR | Beta-lactamase SHV-34 | E. COLI | CHEZ_ECOBD | 21 |
| WT | Beta-lactamase SHV-34 | E. COLI | CHEZ_ECOBD | 75 |
| | Beta-nerve growth factor | HOMO | NGF HUMAN | 41 |
| | | SAPIENS | | |

Analysis of Cleavage Intermediates with Western Blot.

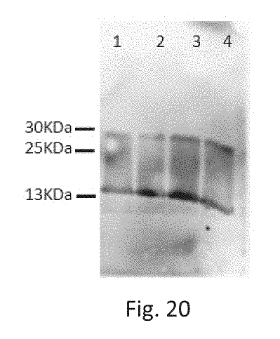

To further control the band of 25 kDa, the inventors decided to carry out an analysis with western blot with antibodies anti-NGF and anti-proNGF. Only non-specific bands can be seen on the membrane incubated with anti-proNGF (data not shown). The membrane was subjected to stripping and then incubated with anti-NGF (FIG. 20). The band of 13 KDa corresponding to mature NGF is clearly present on the membrane for all wells. The antibody detects even a more marked band of 25 KDa in lanes loaded with the proteolytic product of hproNGF wt and a less marked one in those loaded with the proteolytic product of hproNGF wt VSAR. From the results obtained with anti-NGF, it is clear that the band of 25 KDa contains the NGF part for both the hNGF wt and the control and hNGF wt VSAR, albeit with smaller quantities of the/latter. The reason why this band was not detected with anti-proNGF could be attributable to the fact that the peptide used to immunise is none other than the one removed at the N-terminal from trypsin and is therefore not present in the intermediate analysed. The hypothesis, though, is not verifiable, because the anti-proNGF datasheet does not provide details about the epitope recognised by the antibody.

The results obtained from the mass spectrometry analysis and with western blot are conflicting. The analysis with western blot revealed the presence of the NGF portion in the band of 25 kDa present in all three proteolytic products of hproNGF wt, wt VSAR and the control, while the mass spectrometry analysis identified NGF only in the band of 25 kDa obtained by proteolysis of hproNGF wt. The inventors can hypothesize that NGF peptides for hproNGF WT VSAR were not identified due to the sensitivity limit of the detection of the technique. Summing up the Western Blot experiment and the MS analysis, the inventors can conclude that the 25 KDa band was a proNGF cleavage intermediate and that the relative dose of this intermediate is lower in hproNGF WT VSAR proteolytic product with respect to hproNGF WT one.

Given the same conditions (incubation time, temperature, pH and concentration of trypsin), hproNGF wt VSAR is proteolysed more effectively than hproNGF wt.

Human proNGF P61SR100E VSAR.

Extensive tests were carried out by proteolytic cleavage by changing the parameters such as the trypsin:protein weight ratio, temperature and incubation times, in order to find the best conditions for the mutant VSAR proteolysis. Each condition was compared with non-VSAR protein used as the control.

### Proteolytic Cleavage Tests in Small Scale.

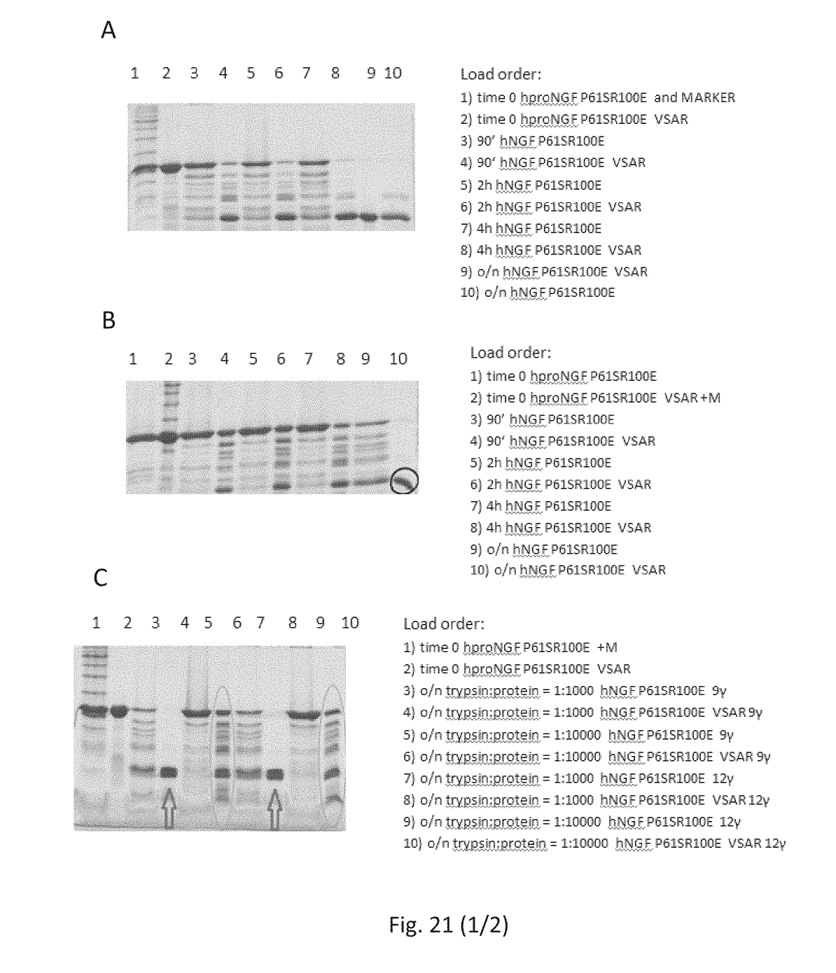

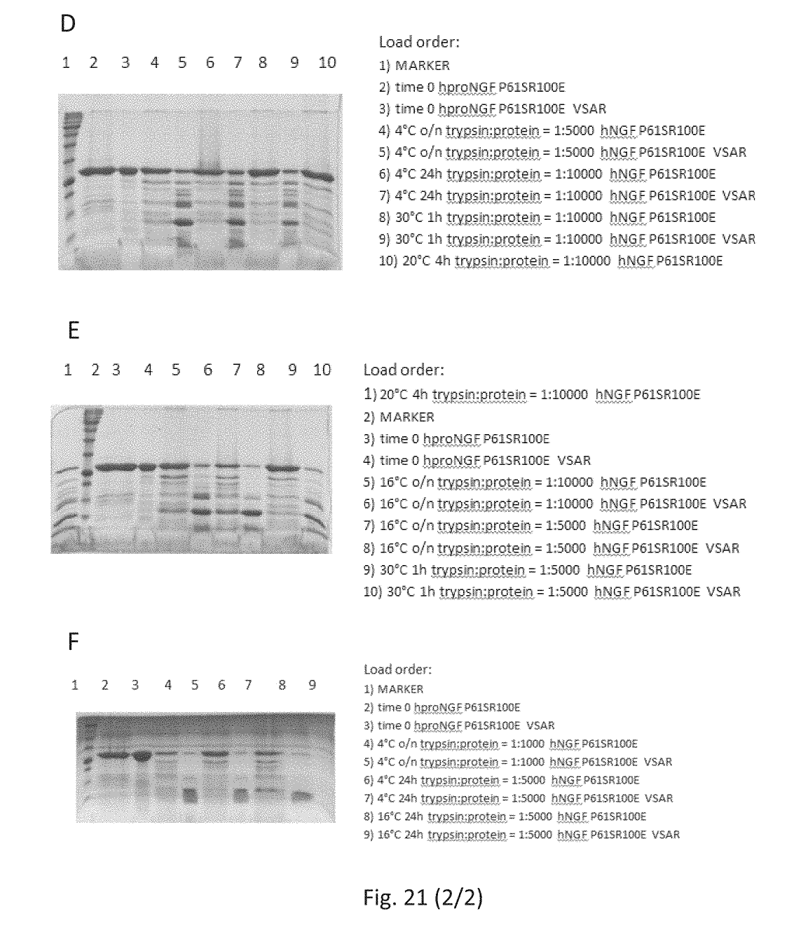

The results of the SDS-PAGE analysis of the proteolytic cleavage product of 120 μg of hproNGF P61SR100E and of mutant hproNGF P61SR100E VSAR under the conditions listed in Table 4 (materials and methods) are shown in FIGS. 21 a-f. The first results obtained by incubation at a temperature of 4° C. show, already after 90 minutes of incubation with trypsin at a trypsin:protein ratio of 1:250 (FIG. 21 a), slight differences between hproNGF P61SR100E and mutant VSAR, which seems to be proteolysed more efficiently, given the same conditions. The intensity and size of the bands corresponding to hproNGF P61SR100E VSAR remaining after proteolysis, in fact, are lower compared with those of the non-mutated control. The differences in response to proteolytic cleavage of the trypsin between hproNGF

P61SR100E and the mutant VSAR increase as the concentration of trypsin used decreases. In fact, after incubation at 4° C. for 18 hours with trypsin at a trypsin:protein ratio of 1:1000, the mutant VSAR is completely proteolysed in NGF (FIG. 21 b, lane 10). In contrast, hproNGF P61SR100E is not cleaved efficiently. In lane 9, besides the presence of the band of 13 kDa corresponding to NGF, the inventors detect even the band at a height of 30 kDa corresponding to proNGF and other bands corresponding to intermediate molecular weight forms generated by partial proteolysis. This confirms that, given the same conditions, the mutant hproNGF P61SR100E VSAR undergoes proteolytic cleavage by trypsin more efficiently than hproNGF P61SR100E. The results of further analyses carried out to search for the best protocol, trying to minimise also the concentration of trypsin, are shown in FIGS. 21 c, d, e, f. FIG. 21 c shows that, at a temperature of 4° C., concentrations of trypsin at a trypsin:protein ratio of 1:10000 are not sufficient for proteolysis of proNGF (lanes 5 and 10), while the inventors have confirmation of the efficiency of proteolysis with trypsin at a trypsin:protein ratio of 1:1000 for 18 hours. Images 21 d, e, f, show the results of the analysis of the proteolytic product of hproNGF P61SR100E and mutant VSAR at low concentrations of trypsin (trypsin:protein ratio of 1:5000 and 1:10000) for different incubation times (1, 18, 24 hours) and temperatures (4° C., 16° C., 20° C. and 30° C.). Among all conditions tested, the ones that allow effective proteolysis at the lowest concentration of trypsin possible are:
1. incubation at 4° C. for 18 hours with trypsin at a trypsin:protein ratio of 1:1000.
2. incubation at 16° C. for 24 hours with trypsin at a trypsin:protein ratio of 1:5000.

### Proteolytic Cleavage Tests in Large Scale.

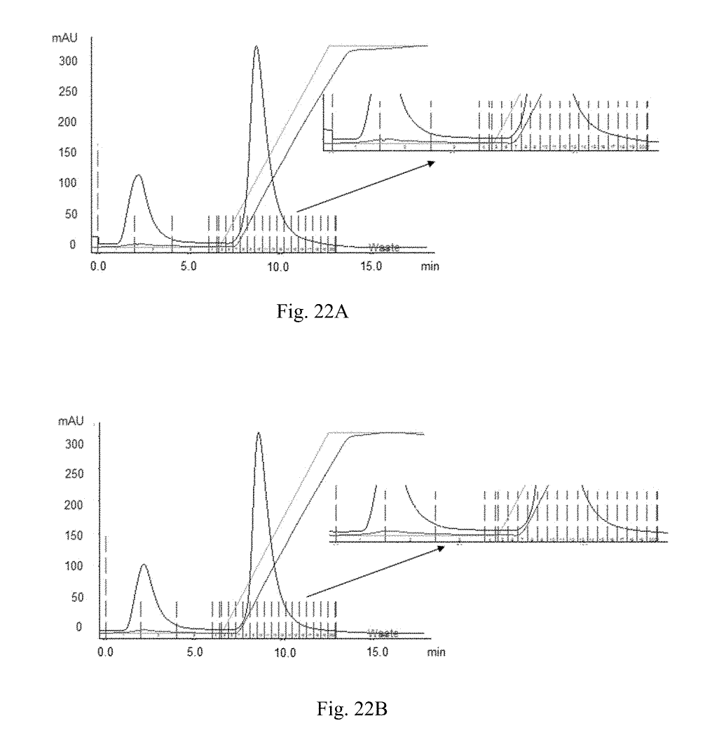

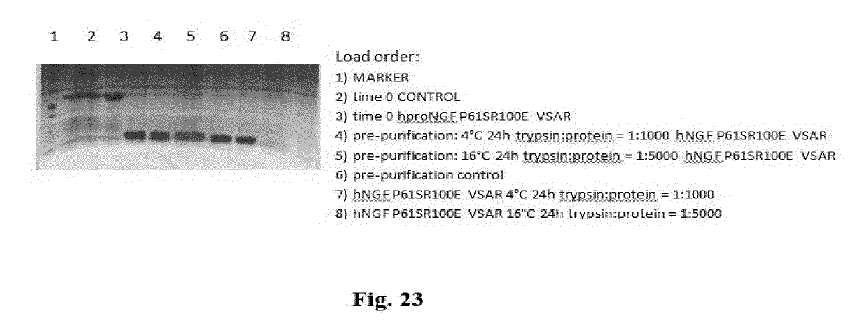

To confirm the results obtained, the cleavage conditions selected as the best from the preliminary analysis on small quantities of protein (incubation at 4° C. for 18 hours with trypsin at a trypsin:protein ratio of 1:1000 and at 16° C. for 24 hours with trypsin at a trypsin:protein ratio of 1:5000) are tested on a larger reaction scale (on 3 mg of hproNGF P61SR100E VSAR), followed by purification and analysis of the peak eluted on SDS-PAGE. The chromatograms obtained by the purification of proteolytic products are shown in FIG. 22. After purification, the fractions eluted corresponding to hNGF P61SR100E VSAR were combined and concentrated. Incubation of 3 mg of hproNGF P61SR100E VSAR at 4° C. for 18 hours with trypsin at a trypsin:protein ratio of 1:1000 produced 2.2 ml of hNGF P61SR100E, whose concentration is 0.40 mg/ml (total 0.88 mg of protein obtained). The yield obtained by proteolysis of 3 mg of proNGF in NGF given by the ratio between the quantity of NGF obtained (0.88 mg) and the quantity of proteolysed proNGF (3 mg) is 0.29. Incubation of 3 mg of hproNGF P61SR100E VSAR at 16° C. for 24 hours with trypsin at a trypsin:protein ratio of 1:5000 produced 2.2 ml of hNGF P61SR100E at a concentration of 0.43 mg/ml (total 0.94 mg of protein). The yield obtained by proteolysis of 3 mg of proNGF in NGF given by the ratio between the quantity of NGF obtained and the quantity of proteolysed proNGF is 0.31. The yield of proteolysis of 3 mg of hproNGF P61SR100E VSAR is about one-third in both cases. To make sure that the proteolytic cleavage has taken place efficiently, the purified proteolytic product is subjected to SDS-PAGE analysis. The gel is loaded with a high quantity of protein (10 μg) in order to detect the possible presence of contaminants at low concentration. The result is shown in FIG. 23. The proteolytic product obtained from proteolytic cleavage in both conditions presents a single band at the height of 13 kDa corresponding to hNGF P61SR100E, demonstrating that the proteolytic cleavage occurred efficiently in both cases.

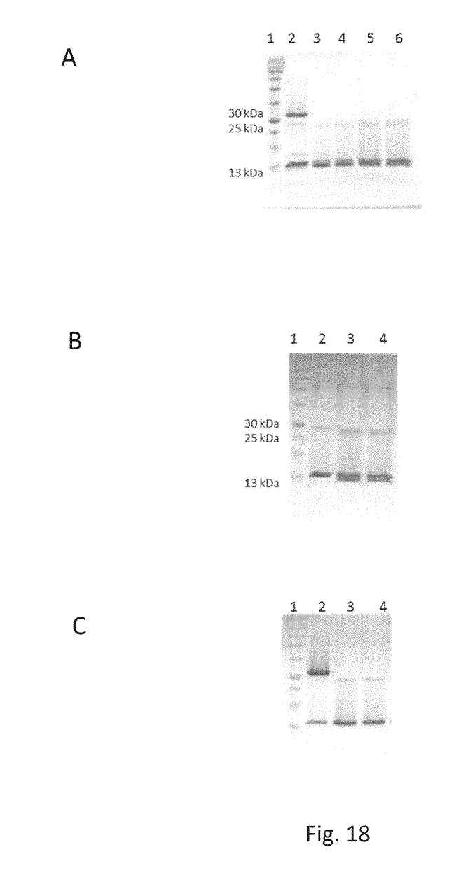

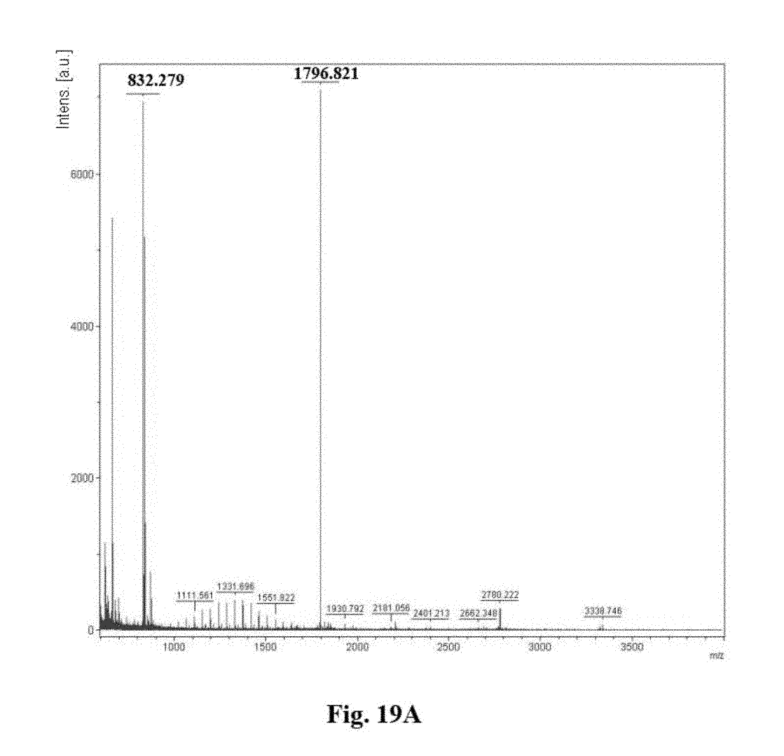

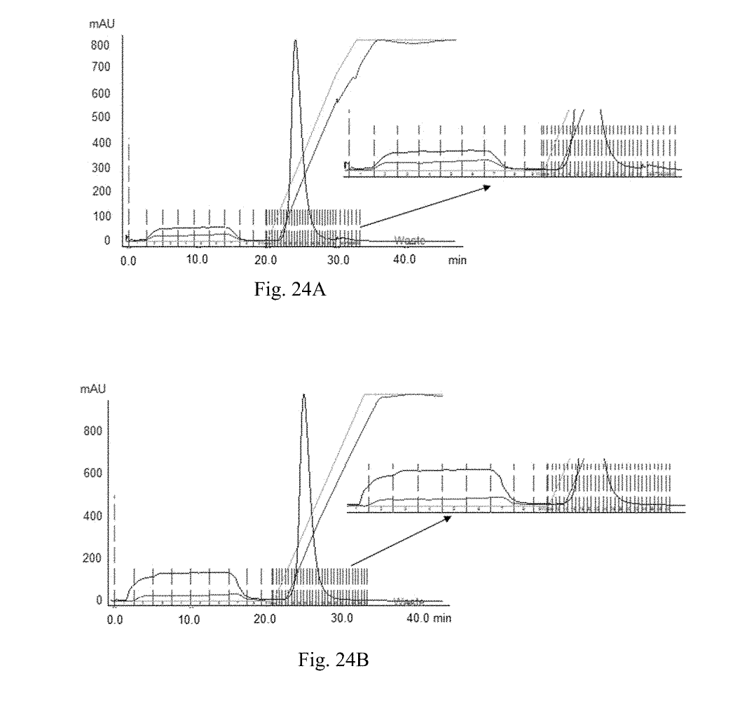

To confirm this result in an even greater reaction scale, the analysis is repeated on 13 mg of hproNGF P61SR100E. The chromatograms are shown in FIG. 24. The fractions eluted from purifications corresponding to hNGF P61SR100E VSAR were combined and concentrated. Incubation of 13 mg of hproNGF P61SR100E VSAR at 4° C. for 18 hours with trypsin at a trypsin:protein ratio of 1:1000 produced 6 ml of hNGF P61SR100E, whose concentration is 0.67 mg/ml (4.02 mg of NGF). The yield of proteolysis given by the ratio between the 4.02 mg of NGF obtained by proteolysis and the 13 mg of proteolysed proNGF is 0.31. Incubation of 13 mg of hproNGF P61SR100E VSAR at 16° C. for 24 hours with trypsin at a trypsin:protein ratio of 1:5000 produced 9 ml of hNGF P61SR100E at a concentration of 0.45 mg/ml (total 4.05 mg of protein). The yield of proteolysis given by the ratio between the 4.05 mg of NGF obtained by proteolysis and the 13 mg of proteolysed proNGF is 0.31. The subsequent analysis by electrophoresis on SDS-PAGE shown in FIGS. 18 and 26 proves that both conditions of proteolysis allow us to obtain a single product. In this case too, the gel was loaded with a high quantity of protein (10 μg) in order to detect the possible presence of contaminants at low concentration. The result obtained from tests on a small scale is therefore confirmed even on larger quantities of protein.

In conclusion the condition chosen for the proteolytic cleavage of the mutant hproNGF P61SR100E VSAR is incubation at 16° C. for 24 hours with trypsin at a trypsin:protein ratio of 1:5000. This protocol allows an efficient proteolysis using the lowest quantity of enzyme possible.

The inventors can conclude that the mutations Arg101Val and Lys103Ala on the consensus sequence of the proteolytic furin enzyme have contributed to a sharp improvement in the cleavage protocol for the double mutant hproNGF P61SR100E. With respect to the conditions used previously for the hproNGF P61SR100E not VSAR, in fact, the inventors obtained, given the same cleaved product:
1. a decrease in the concentrations of trypsin needed for reaction, with cost savings (trypsin:hproNGF P61SR100E VSAR ratio of 1:5000 compared to the trypsin:hproNGF P61SR100E ratio of 1:250 used previously)
2. incubation at low temperature (16° C. compared to 30° C.), ideal to avoid unwanted proteolysis or destabilisation of the protein.

### NGF-Dependent Cell Proliferation Assay on TF1 Cells

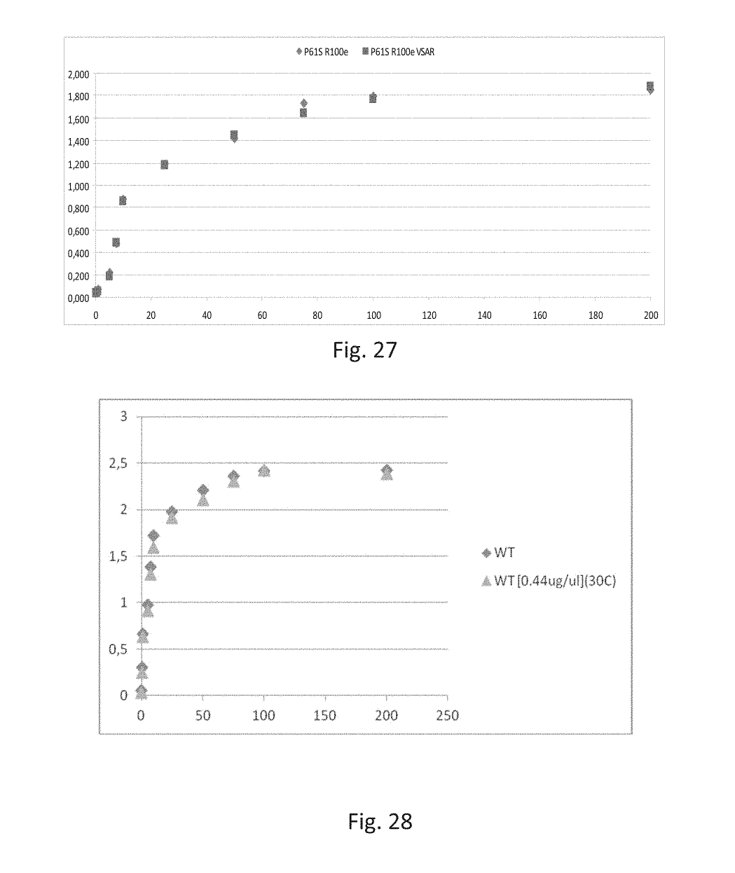

To control the bioactivity of NGF, the inventors used the quantitative assay of cell proliferation with TF1 cells. The TF1 cells are a human hematopoietic cellular line that expresses the Trka receptor of NGF but not p75NTR. Administration of the exogenous NGF induces autophosphorylation of the TrkA receptor, triggering a cell proliferation signal. For their ability to respond to exogenous NGF by proliferating, they are a good quantitative assay to assess the functionality of hNGF produced in the laboratory. Depending on the concentration, NGF produces a dose/response curve of viability. In the presence of a standard, in fact, it is possible to assess the bioactivity of each batch of protein produced. In this case, NGF P61SR100E derived from mutant VSAR should not display differences with the same non-VSAR mutant, as the mutation is present on the precursor part that is eliminated in the proteolytic cleavage. The hypothesis is correct: as shown in FIG. 27, the dose/response curve of NGF produced by hproNGF VSAR overlaps the one of the standard.

hNGF wt was also tested on the TF1 cells. FIG. 28 shows the dose/response curve obtained with hNGF wt obtained from proteolytic cleavage of hproNGF wt VSAR with trypsin at a trypsin:hproNGF ratio of 1:500 for 2 hours at 30° C., which had been identified as the best condition. In this case too, the dose/response curve of NGF produced by mutant VSAR overlaps the one of the standard.

In conclusion, the mutants of the precursor of NGF, hproNGF wt VSAR and hproNGF P61SR100E VSAR were produced by site-directed mutagenesis of the sequence Arg-Ser-Lys-Arg (positions 101-104), consensus of furin endoprotease, in order to optimise the cleavage protocol with trypsin to obtain mature NGF. The mutants were obtained, expressed and purified by obtaining a yield of proNGF almost unchanged compared to the corresponding non-mutated controls. Among the various cleavage protocols tested, the best performing are:
incubation at 16° C. for 24 hours with trypsin at a trypsin:protein ratio of 1:5000 for hproNGF P61SR100E VSAR.
incubation at 30° C. for 2 hours with trypsin at a trypsin:protein ratio of 1:500 for hproNGF wt VSAR.

The yield of proteolysis of the mutant hproNGF wt VSAR compared with the non-mutated control was similar for the wt, around 30% for both. For the double mutant VSAR, instead, in comparison with the data previously collected in the laboratory, the yield of proteolytic cleavage (about 30%) appears lower in the experiments that I carried out: the cleavage yield for hproNGF P61SR100E is around 40%.

For hNGF P61SR100E VSAR, the desired results were achieved: the proNGF is completely cleaved, using smaller quantities of trypsin, with significant cost savings. Unlike the protocol in use, the reaction occurs at low temperature, ideal condition to avoid unwanted proteolysis or destabilisation of the protein.

With hproNGF wt VSAR, despite the various protocols explored, the result was not fully achieved, as there continues to be the presence of a cleavage intermediate in the preparations tested. It should be noted, though, that hproNGF wt VSAR is cleaved however more efficiently than the corresponding non-mutated one, with a sharply lower intermediate concentration. The partial result obtained, furthermore, is to be considered irrelevant to the aim pursued: the proNGF wt represented only a study control, as the protein intended for therapeutic use is the painless mutant.

In the present invention a crucial aspect of the production process of mutant hNGF P61SR100E intended for therapeutic use has been improved. The standardisation of proteolytic cleavage will help speed up transfer of the process for a protein production under GMP.

### Site Specific Mutagenesis of Single Mutants

The point mutations Val101Arg e Ala103Lys were correctly inserted in the plasmids pET11a, containing the ORF hproNGF P61S VSAR and hproNGF R100E VSAR, and assessed by sequencing the plasmid DNA.

The E. coli strain Rosetta (DE3)pLysS was transformed with the obtained plasmids pet11a hproNGF P61S VSAR and pet11a hproNGF R100E VSAR.

### Expression and Purification of VSAR Single Mutants

hproNGF P61S VSAR and hproNGF R100E VSAR were expressed in E. coli Rosetta (DE3)pLysS and purified as previously described (Malerba et al 2015). The yields were compared to those obtained from the non VSAR proNGF (Malerba et al, 2015). Comparing all the VSAR mutants yields, it is clear that only hproNGFP61SR100E VSAR and hproNGF WT VSAR kept the same expression level, as their non-VSAR version, while the VSAR versions of the single mutants express much less (table 6).

TABLE 6
Yields of the purified hproNGF WT VSAR, hproNGFP61SR100E
VSAR, hproNGF P61S VSAR and hproNGF R100E VSAR.

| | Yield ProNGF | Yield proNGF VSAR |
|---|---|---|
| Mutant | (mg/L) | (mg/L) |
| P61S | 24 | 14 |
| R100E | 9.3 | 3 |
| P61S R100E | 8.3 | 7.5 |
| WT | 35.0 | 33.8 |

Yields of the purified hproNGF WT VSAR, hproNGFP61SR100E VSAR, hproNGF P61S VSAR and hproNGF R100E VSAR, expressed in E. coli Rosetta (DE3)pLysS. The yields were compared to those obtained from the corresponding non VSAR proNGF (Malerba et al, 2015).

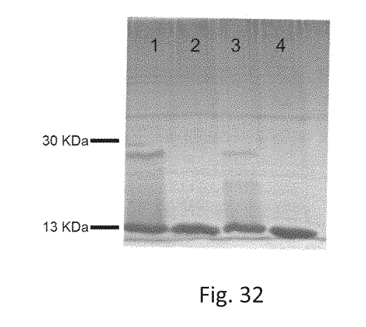

hNGF P61SR100E was also analyzed by Maldi TOF Mass Spectrometry. As evident from the FIG. 32, a unique species is present, of the expected molecular weight.

### Conclusion

hNGF P61SR100E VSAR exhibited the same yield as for hNGF P61SR100E (for both the proNGF and NGF forms). The VSAR mutation allowed a less expensive protocol, due to the significantly lower concentration of trypsin, and assured a homogenous cleavage (verified by Mass Spectrometry), unlike the non VASR mutants. A side by side comparison of hpro NGF P61SR100E VSAR versus hpro NGF WT VSAR cleavage is shown below (FIG. 8), showing that the cleavage of of hpro NGF P61SR100E VSAR is much more efficient of that of hpro NGF WT VSAR.

VSAR method appears to depend very much on the sequences in the mature NGF moiety. Indeed, an intermediate of 25 KDa was observed in all the conditions tested, while this was not the case for the hpro NGF P61SR100E VSAR double mutant (see above). hproNGF WT VSAR was cleaved more efficiently than hproNGF WT. Therefore, it is particularly important and unexpected that hNGFP61SR100E VSAR was cleaved completely and homogeneously (see above). Therefore the 61/100 VSAR mutant performs better than the 61/100 and better than the NGF wt VSAR. This is not expected from the prior knowledge.

hproNGF P61S VSAR and hproNGF R100E VSAR Proteolytic Cleavage

In order to compare the result obtained with hproNGF WT VSAR and hproNGF P61SR100E, experiments with single mutants (hproNGF P61S VSAR and hproNGF R100E VSAR) were carried out.

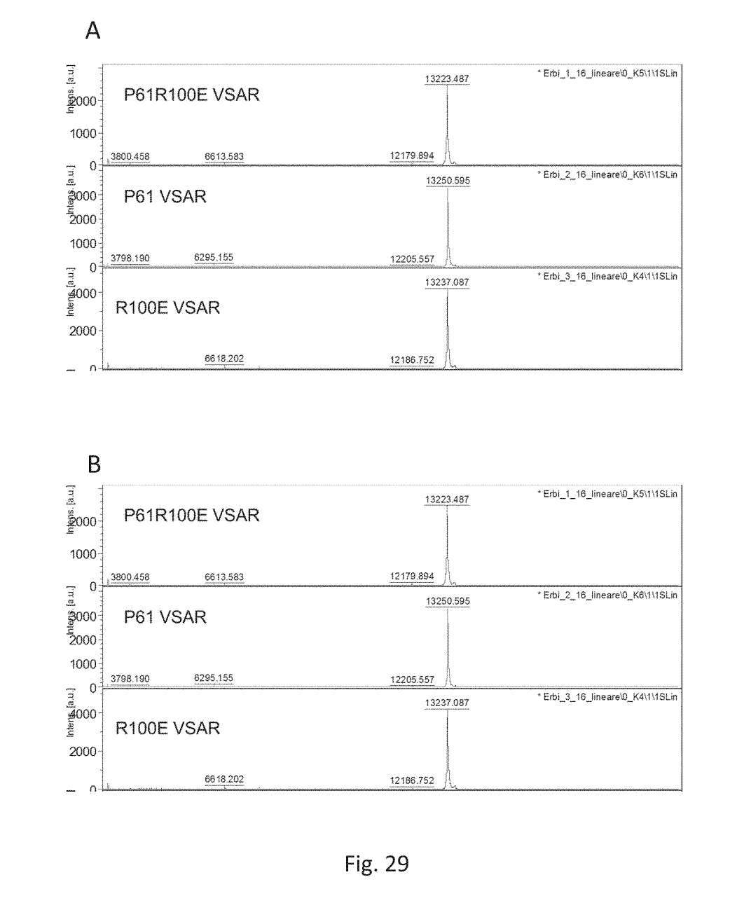

120 μg of hproNGF P61SR100E, hproNGF P61S VSAR and hproNGF R100E VSAR (concentration 0.6 mg/ml) were incubated with trypsin (promega) at the condition indicated in table 4 and then analyzed directly by Mass Spectrometry. The Mass Spectrometry analysis showed different species at different molecular weight (FIG. 29). The samples treated at 16° C. for 24 h with trypsin:protein=1:5000 (FIG. 29, panel A) showed similar results: two species, despite at different ratios, probably corresponding to the expected mature NGF, and to the desocta form. The samples treated at 4° C. for 24 h with trypsin:protein=1:1000 (FIG. 29, panel B) showed, beside these two species, also an intermediate at 16 KDa, but only for hproNGF P61SR100E and hproNGF P61S VSAR.

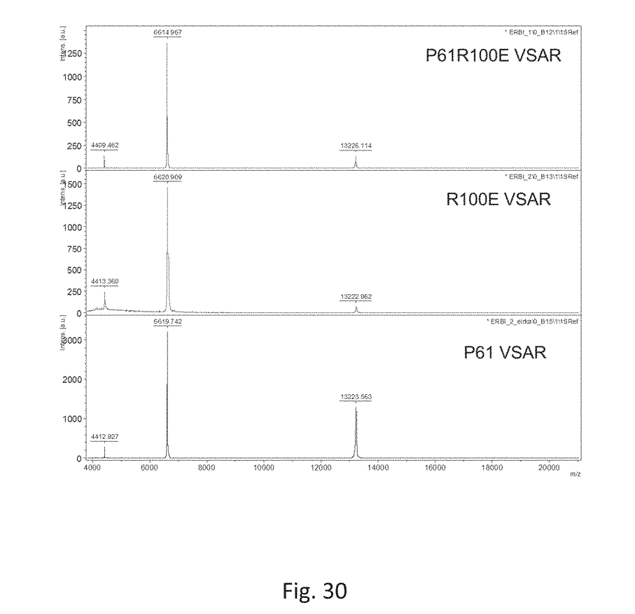

The best condition (16° C. 24 h trypsin:protein=1:5000) was chosen to carry out the cleavage experiment in larger scale, with 3 mg of hproNGF P61SR100E, hproNGF P61S VSAR and hproNGF R100E, as described in Methods. As evident from the FIG. 30, upon purification, only the correct species corresponding to mature NGF is revealed by Mass Spectrometry, for all the mutants tested.

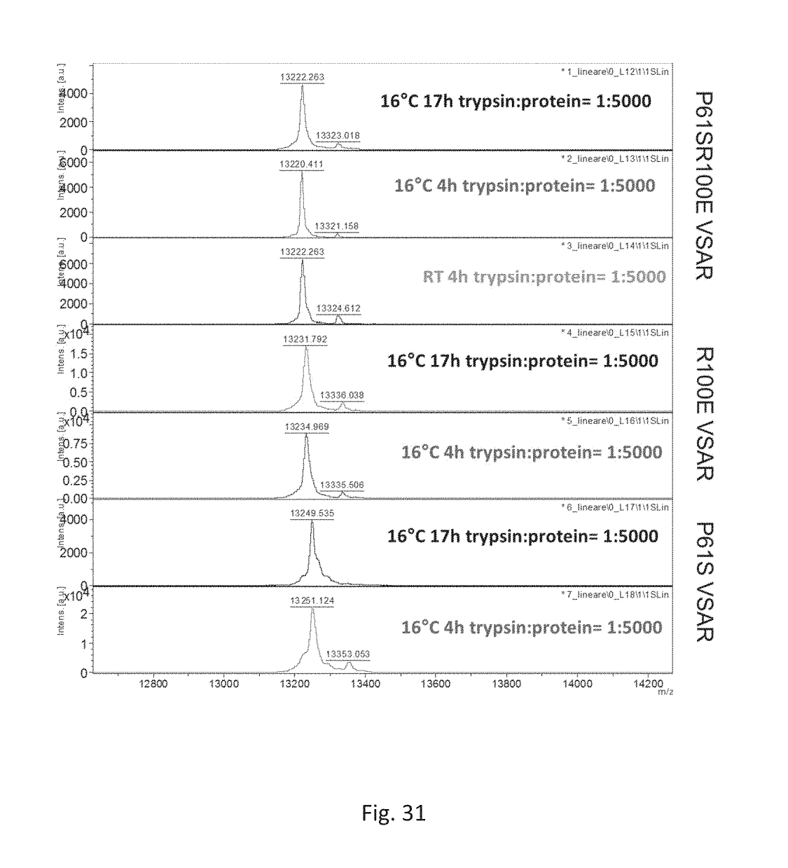

In order to check possible differences between the mutants, other cleavage conditions were explored, listed in table 5. The cleavage products were purified, as described, and analyzed by Mass Spectrometry. As evident from the FIG. 31, all the protocols gave rise to a main species and a contaminant.

VSAR mutation has the same effect on proteolytic cleavage for single and double mutants. Indeed, the mass spectrometry results showed the same molecular species, for the double and single mutants, if treated under the same cleavage conditions. The best cleavage protocol was confirmed to be 16° C., 24 h, trypsin:protein=1:5000. Surprisingly, also minor differences in the protocol gave rise to other molecular species, and to a heterogeneous cleavage product, as always seen for the WT protein, under all conditions.

Side by Side hproNGF P61SR100E VSAR Versus hproNGF WT VSAR Comparison

3 mg of hproNGF P61SR100E and hproNGF WT (concentration 0.6 mg/ml) were digested in parallel, by following these two protocol:

1) Room Temperature, 18 h, trypsin:protein=1:10000 (WO 2013/092776 conditions)
2) 16° C., 24 h, trypsin:protein=1:5000 (inventors' best conditions)
and purified. The positive fractions were collected and concentrated, as described in methods. 10 μg of each purified sample were run on a SDS PAGE (15% Polyacrylamide) and the colored by EzWay™ Protein-Quick Blue Staining Solution (KOMABIOTECH). hproNGF P61SR100E and hproNGF WT in both the conditions gave rise to the mature NGF, as evident from the band at 13 kDa. In the gel the lanes 1 and 3, corresponding to hproNGF WT, another band at about 25 kDa is evident, despite its intensity is lower in the condition 2 (FIG. 32).

The side-by-side comparison demonstrates that, unexpectedly, hproNGF P61SR100E VSAR is cleaved much more precisely and more efficiently than hproNGF WT VSAR. Both the SDS-PAGE and the Mass Spectrometry analyses demonstrated that the optimized cleavage conditions of hproNGF P61SR100E VSAR gave rise to a homogenous and unique molecular species, at the correct molecular weight, unlike the WT hproNGF VSAR protein.

### REFERENCES

Paoletti F, Covaceuszach S, Konarev P V, Gonfloni S, Malerba F, Schwarz E, Svergun D I, Cattaneo A, Lamba D. Intrinsic structural disorder of mouse proNGF. Proteins. 2009 June; 75(4):990-1009
Malerba F, Paoletti F, Bruni Ercole B, Materazzi S, Nassini R, Coppi E, Patacchini R, Capsoni S, Lamba D, Cattaneo A. Functional Characterization of Human ProNGF and NGF Mutants: Identification of NGF P61SR100E as a “Painless” Lead Investigational Candidate for Therapeutic Applications. PLoS One. 2015 Sep. 15; 10(9):e0136425.
Chevalier S, Praloran V, Smith C, MacGrogan D, Ip N Y, Yancopoulos G D, Brachet P, Pouplard A, Gascan H. Expression and functionality of the trkA proto-oncogene product/NGF receptor in undifferentiated hematopoietic cells. Blood. 1994 Mar. 15; 83(6):1479-85.
LEVI-MONTALCINI R, MEYER H, HAMBURGER V. In vitro experiments on the effects of mouse sarcomas 180 and 37 on the spinal and sympathetic ganglia of the chick embryo. Cancer Res. 1954 January; 14(1):49-57.
Burnham P, Raiborn C, Varon S. Replacement of nerve-growth factor by ganglionic non-neuronal cells for the survival in vitro of dissociated ganglionic neurons. Proc Natl Acad Sci USA. 1972 December; 69(12):3556-6
Holland D R, Cousens L S, Meng W, Matthews B W. Nerve growth factor in different crystal forms displays structural flexibility and reveals zinc binding sites. J Mol Biol. 1994 Jun. 10; 239(3):385-400.

1. A pro-NGF mutant wherein the protease cleavage site R_{A}SKR_{B} is substituted at least at position RA and K by a non-basic amino acid said pro-NGF mutant further comprising at least one mutation selected from P165S or R204E.

2. The pro-NGF mutant according to claim 1 wherein the non-basic amino acid is selected from the group consisting of Alanine, Glycine, Valine, Serine, Threonine, Methionine, Tyrosine, Histidine, Asparagine, Aspartic Acid, Glutamine, Glutamic Acid, Phenylalanine, Isoleucine, Leucine, Tryptophan, Cysteine, and Proline.

3. The pro-NGF mutant according to claim 1 wherein the protease cleavage site has the sequence VSAR.

4. The pro-NGF mutant according to claim 1 comprising both mutations P165S and R204E.

5. The pro-NGF mutant according to claim 1 for use in a method to produce a NGF mutant.

6. The pro-NGF mutant according to claim 5 for use in a method to produce a NGF mutant, said NGF mutant comprising at least one of the mutation selected from NGF P61S or R100E.

7. A method for the production of a NGF mutant comprising the steps of incubating the pro-NGF mutant as defined in claim 1 with trypsin.

8. The method according to claim 7 wherein the incubation time is about 24 hours.

9. The method according to claim 7 wherein the incubation temperature is between about 4° to about 16° C.

10. The method according to claim 7 wherein the weight ratio trypsin:pro-NGF is 1:5000, the incubation temperature is about 16° C. and the incubation time is about 24 hours and the proNGF mutant concentration is about 0.6 mg/ml.
